(19) United States
(12) Patent Application Publication
(10) Pub. No.: US 2020/0113636 A1
(43) Pub. Date: Apr. 16, 2020

(54) ROBOTICALLY-ASSISTED SURGICAL DEVICE, ROBOTICALLY-ASSISTED SURGERY METHOD, AND SYSTEM

(71) Applicant: Ziosoft, Inc., Tokyo (JP)

(72) Inventors: Shusuke CHINO, Tokyo (JP); Tsuyoshi NAGATA, Tokyo (JP); Yutaka KARASAWA, Tokyo (JP); Shinichiro SEO, Tokyo (JP)

(21) Appl. No.: 16/599,364

(22) Filed: Oct. 11, 2019

(30) Foreign Application Priority Data
Oct. 11, 2018 (JP) 2018-192936

(51) Int. Cl. A61B 34/30; A61B 90/00

(52) U.S. Cl. CPC A61B 34/30; A61B 2034/105; A61B 90/37

(57) ABSTRACT

A robotically-assisted surgical device that assists minimally invasive robotic surgery with a surgical robot is configured to acquire volume data of a non-pneumoperitoneum state of a subject, perform a pneumoperitoneum simulation on the volume data of the non-pneumoperitoneum state to generate deformation information including movement of at least one point in the volume data of the non-pneumoperitoneum state caused by pneumoperitoneum, generate 3D data of a first virtual pneumoperitoneum state based on the volume data of the non-pneumoperitoneum state and the deformation information, derive a first planned position in the 3D data of the first virtual pneumoperitoneum state, derive a second planned position in the volume data of the non-pneumoperitoneum state based on the first planned position in the first virtual pneumoperitoneum state and the deformation information, and visualize the volume data of the non-pneumoperitoneum state with an annotation of information indicating the second planned position.

### CROSS-REFERENCE TO RELATED APPLICATIONS

This application is based on and claims priority under 35 USC 119 from Japanese Patent Application No. 2018-192936 filed on Oct. 11, 2018, the contents of which are incorporated herein by reference.

### TECHNICAL FIELD

The present disclosure relates to a robotically-assisted surgical device that assists robotic surgery with a surgical robot, a robotically-assisted surgery method, and a system.

### BACKGROUND ART

In the related art, when minimally invasive robotic surgery is operated using a surgical robot, a port for inserting forceps into the body of a patient being operated is pierced. The position of the port is approximately determined depending on a surgical procedure, but the optimal position thereof has yet to be established.

US2014/0148816A discloses port placement planning. Specifically, a surgical port placement system disclosed in US2014/0148816A generates a surgical port placement model based upon a plurality of parameter sets associated with a plurality of past surgical procedures, receives a given parameter set for a given surgical procedure including physical characteristics of a given patient, and plans at least one port position for the given patient for the given surgical procedure based upon the given parameter set and the surgical port placement model.

### SUMMARY OF INVENTION

The present disclosure provides a robotically-assisted surgical device capable of planning a port position on a body surface of a subject on which pneumoperitoneum is to be performed before pneumoperitoneum, a robotically-assisted surgery method, and a system.

According to one aspect of the present disclosure, a robotically-assisted surgical device that assists minimally invasive robotic surgery with a surgical robot includes a processing unit and a display unit. The processing unit is configured to acquire volume data of a non-pneumoperitoneum state of a subject, perform a pneumoperitoneum simulation on the volume data of the non-pneumoperitoneum state to generate first deformation information including movement of at least one point in the volume data of the non-pneumoperitoneum state, the movement being caused by pneumoperitoneum, generate 3D data of a first virtual pneumoperitoneum state based on the volume data of the non-pneumoperitoneum state and the first deformation information, derive a first planned position that is a planned position of a port on a body surface of the subject in the 3D data of the first virtual pneumoperitoneum state, derive a second planned position that is a planned position of a port on the body surface of the subject in the volume data of the non-pneumoperitoneum state based on the first planned position in the first virtual pneumoperitoneum state and the first deformation information, and cause the display unit to visualize the volume data of the non-pneumoperitoneum state with an annotation of information indicating the second planned position.

According to one aspect of the present disclosure, a robotically-assisted surgery method for assisting minimally invasive robotic surgery with a surgical robot includes: acquiring volume data of a non-pneumoperitoneum state of a subject; performing a pneumoperitoneum simulation on volume data of the non-pneumoperitoneum state to generate 3D data of a virtual pneumoperitoneum state; generating deformation information representing a corresponding relationship between respective points in the volume data and respective points in the 3D data based on the volume data of the non-pneumoperitoneum state and the 3D data of the virtual pneumoperitoneum state; deriving a first planned position that is a planned position of a port on a body surface of the subject in the 3D data of the virtual pneumoperitoneum state; deriving a second planned position that is a planned position of a port on the body surface of the subject in the volume data of the non-pneumoperitoneum state based on the first planned position in the virtual pneumoperitoneum state and the deformation information; and causing a display unit to visualize the volume data of the non-pneumoperitoneum state with an annotation of information indicating the second planned position.

According to one aspect of the present disclosure, a system includes: a surgical robot; and a robotically-assisted surgical device that assists minimally invasive robotic surgery with the surgical robot and includes a processing unit and a display unit. The processing unit is configured to acquire volume data of a non-pneumoperitoneum state of a subject, perform a pneumoperitoneum simulation on the volume data of the non-pneumoperitoneum state to generate first deformation information including movement of at least one point in the volume data of the non-pneumoperitoneum state, the movement being caused by pneumoperitoneum, generate 3D data of a first virtual pneumoperitoneum state based on the volume data of the non-pneumoperitoneum state and the first deformation information, derive a first planned position that is a planned position of a port on a body surface of the subject in the 3D data of the first virtual pneumoperitoneum state, derive a second planned position that is a planned position of a port on the body surface of the subject in the volume data of the non-pneumoperitoneum state based on the first planned position in the first virtual pneumoperitoneum state and the first deformation information, and cause the display unit to visualize the volume data of the non-pneumoperitoneum state with an annotation of information indicating the second planned position.

The present disclosure provides a robotically-assisted surgical device capable of suppressing deterioration in the workability of robotic surgery by a surgical robot, a robotically-assisted surgery method, and a system.

### BRIEF DESCRIPTION OF DRAWINGS

FIG. 1 is a block diagram illustrating a hardware configuration example of a robotically-assisted surgical device according to a first embodiment;

FIG. 2 is a block diagram illustrating a functional configuration example of the robotically-assisted surgical device;

FIG. 3 is a view illustrating examples of MPR images of the abdomen before and after performing a pneumoperitoneum simulation;

FIG. 4 is a view illustrating a measurement example of a port position of a pre-pierced port;

FIG. 5A is a view illustrating a first placement planning example of port positions placed on a body surface of a subject;

FIG. 5B is a view illustrating a second placement planning example of port positions placed on the body surface of the subject;

FIG. 5C is a view illustrating a third placement planning example of port positions placed on the body surface of the subject;

FIG. 6 is a view illustrating an example of a positional relationship between the subject, ports, trocars, and robot arms during robotic surgery;

FIG. 7 is a flowchart illustrating an example of a procedure of a port position simulation by the robotically-assisted surgical device;

FIG. 8 is a flowchart illustrating an operation example when a port position score is calculated by the robotically-assisted surgical device:

FIG. 9 is a view illustrating an example of working areas determined based on port positions;

FIG. 10 is a view illustrating a movement example of a port position before and after a pneumoperitoneum simulation;

FIG. 11 is a flowchart illustrating an operation example when the port position is derived by the robotically-assisted surgical device before pneumoperitoneum;

FIG. 12 is a view illustrating the handling of non-uniform expansion of the subject in the pneumoperitoneum simulation;

FIG. 13 is a view illustrating the handling of the non-uniform expansion of the subject in the pneumoperitoneum simulation;

FIG. 14 is a view illustrating the handling of errors corresponding to the amount of gas in the pneumoperitoneum simulation;

FIG. 15 is a view illustrating a corresponding positional relationship on body surfaces of different virtual pneumoperitoneum states; and

FIG. 16 is a flowchart illustrating a derivation procedure of allowable error information by the robotically-assisted surgical device.

### DESCRIPTION OF EMBODIMENTS

Hereinafter, embodiments of the present disclosure will be described using the drawings.

In the present disclosure, a robotically-assisted surgical device that assists minimally invasive robotic surgery with a surgical robot includes a processing unit and a display unit. The processing unit is configured to acquire volume data of a non-pneumoperitoneum state of a subject, perform a pneumoperitoneum simulation on the volume data of the non-pneumoperitoneum state to generate first deformation information including movement of at least one point in the volume data of the non-pneumoperitoneum state, the movement being caused by pneumoperitoneum, generate 3D data of a first virtual pneumoperitoneum state based on the volume data of the non-pneumoperitoneum state and the first deformation information, derive a first planned position that is a planned position of a port on a body surface of the subject in the 3D data of the first virtual pneumoperitoneum state, derive a second planned position that is a planned position of a port on the body surface of the subject in the volume data of the non-pneumoperitoneum state based on the first planned position in the first virtual pneumoperitoneum state and the first deformation information, and cause the display unit to visualize the volume data of the non-pneumoperitoneum state with an annotation of information indicating the second planned position.

According to the present disclosure, even when an elevation degree in an abdominal cavity of the subject varies or the figure of elevation in the abdominal cavity varies, the robotically-assisted surgical device can associate a port position that is planned after pneumoperitoneum with a position corresponding to a port position before pneumoperitoneum. In addition, by associating the port positions before and after pneumoperitoneum with each other, the robotically-assisted surgical device can plan, before pneumoperitoneum, a position of a port on the body surface of the subject on which pneumoperitoneum is to be performed. Accordingly, the robotically-assisted surgical device can plan a position of a port on the subject of the non-pneumoperitoneum state before the start of the operation and can plan a position of a port without considering an operative duration. Accordingly, the robotically-assisted surgical device can reduce a mental burden relating to port position planning on an operator. In addition, since the port position is planned before the operation to omit port position planning during operation, the operative duration can be reduced, and a physical burden on the subject can also be reduced. In addition, by planning the port position before pneumoperitoneum, the user can measure the port position using a ruler or the like and place the port position before pneumoperitoneum. In this case, the body surface of the subject before pneumoperitoneum is flatter than that after pneumoperitoneum. Therefore, the user easily measures the port position by putting a ruler on the body surface and easily place the port position.

### (Circumstances for Achievement of Aspect of Present Disclosure)

In robotic surgery, pneumoperitoneum is performed in many cases. During pneumoperitoneum, gas (for example, carbon dioxide) is injected into the abdominal cavity such that a working space is secured in the abdominal cavity. A position corresponding to the subject moves before and after pneumoperitoneum. Therefore, in many cases, a port position on the body surface of the subject is planned in consideration of a state of pneumoperitoneum where an actual treatment is performed in surgery. Since the degree of elevation of abdominal wall varies depending on the pneumoperitoneum state, the figure of elevation in the abdominal cavity can also vary depending on the subject. Therefore, it is difficult to associate a port position on the body surface of the subject that is planned after pneumoperitoneum with a position corresponding to a port position on the body surface of the subject before pneumoperitoneum.

In addition, pneumoperitoneum on a subject represents the start of surgery on the subject. Therefore, when a long period of time is required for pneumoperitoneum and port position planning, the total surgery duration is extended. Accordingly, during placement of a port position after pneumoperitoneum, a mental burden on an operator is large, and a burden on a patient is also large.

In the following embodiments, a robotically-assisted surgical device that can plan a port position on a body surface of a subject on which pneumoperitoneum is to be performed before the pneumoperitoneum, a robotically-assisted surgery method, and a system will be described.

### First Embodiment

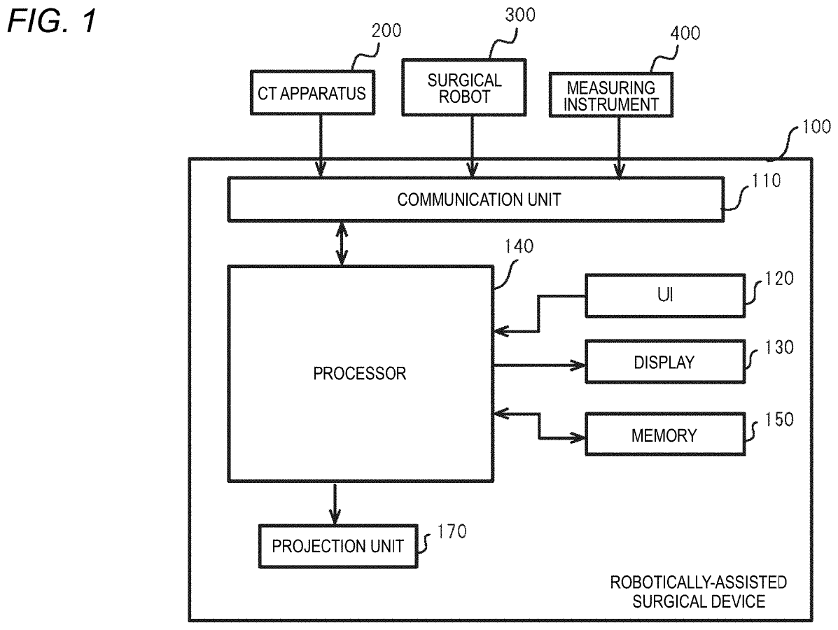

FIG. 1 is a block diagram illustrating a configuration example of a robotically-assisted surgical device 100 according to a first embodiment. The robotically-assisted surgical device 100 assists robotic surgery with a surgical robot 300 and performs, for example, a preoperative simulation, an intraoperative simulation, and intraoperative navigation.

The surgical robot 300 includes a robot operation terminal, a robot main body, and an image display terminal.

The robot operation terminal includes a hand controller or a foot switch manipulated by an operator. The robot operation terminal operates a plurality of robot arms AR provided in the robot main body according to a manipulation of the hand controller or the foot switch by the operator. In addition, the robot operation terminal includes a viewer. The viewer may be a stereo viewer and may merge images input through an endoscope to display a 3D image. A plurality of robot operation terminals may be present such that a plurality of operators operate the plurality of robot operation terminals to perform robotic surgery.

The robot main body includes: a plurality of robot arms for performing robotic surgery; and an end effector EF (forceps, an instrument) as a surgical instrument that is mounted on the robot arm AR.

The robot main body of the surgical robot 300 includes four robot arms AR including: a camera arm on which an endoscope camera is mounted; a first end effector arm on which an end effector EF operated by a right-hand controller of the robot operation terminal is mounted; a second end effector arm on which an end effector EF operated by a left-hand controller of the robot operation terminal is mounted; and a third end effector arm on which an end effector EF for replacement is mounted. Each robot arm AR includes a plurality of joints and includes a motor and an encoder corresponding to each joint. Each robot arm AR has at least 6 degrees of freedom and preferably 7 or 8 degrees of freedom, operates in a 3D space, and may be movable in each direction in the 3D space. The end effector EF is an instrument that actually comes into contact with a treatment target in a subject PS during robotic surgery, and can perform various treatments (for example, gripping, dissection, exfoliation, or suture).

Examples of the end effector EF include gripping forceps, exfoliating forceps, an electric knife, and the like. A plurality of different end effectors EF may be prepared for respective functions. For example, in robotic surgery, a treatment of dissecting a tissue with one end effector EF while holding or pulling the tissue with two end effectors EF may be performed. The robot arm AR and the end effector EF may operate based on an instruction from the robot operation terminal.

The image display terminal includes a monitor, a controller for processing an image captured by a camera of an endoscope to display the image on a viewer or a monitor, and the like. The monitor is checked by, for example, an assistant of robotic surgery or a nurse.

The surgical robot 300 receives a manipulation of the hand controller or the foot switch of the robot operation terminal by the operator, controls the operation of the robot arm AR or the end effector EF of the robot main body, and performs robotic surgery in which various treatments are performed on the subject PS. In robotic surgery, laparoscopic surgery is performed in the subject PS.

In robotic surgery, a port PT is pierced on the body surface of the subject PS, and pneumoperitoneum is performed through the port PT. In pneumoperitoneum, carbon dioxide may be injected to inflate the abdominal cavity of the subject PS. In the port PT, a trocar TC may be provided. The trocar TC includes a valve and maintains the inside of the subject PS to be airtight. In addition, in order to maintain the airtight state, air (for example, carbon dioxide) is intermittently introduced into the subject PS.

The end effector EF (shaft of the end effector EF) is inserted into the trocar TC. The valve of the trocar TC is opened during insertion of the end effector EF and is closed during the separation of the end effector EF. The end effector EF is inserted from the port PT through the trocar TC such that various treatments are performed according to the surgical procedure. Robotic surgery may be applied to not only laparoscopic surgery in which the surgery target is the abdomen but also arthroscopic surgery in which the surgery target includes a region other than the abdomen.

As illustrated in FIG. 1, the robotically-assisted surgical device 100 includes a communication unit 110, a user interface (UI) 120, a display 130, a processor 140, and a memory 150. The UI 120, the display 130, and the memory 150 may be included in the robotically-assisted surgical device 100 or may be provided separately from the robotically-assisted surgical device 100.

A CT (Computed Tomography) scanner 200 is connected to the robotically-assisted surgical device 100 through the communication unit 110. The robotically-assisted surgical device 100 acquires volume data from the CT apparatus 200 and processes the acquired volume data. The robotically-assisted surgical device 100 may be configured by a PC (Personal Computer) and software installed on the PC. The robotically-assisted surgical device 100 may be configured as a part of the surgical robot 300.

The surgical robot 300 is connected to the robotically-assisted surgical device 100 through the communication unit 110. The robotically-assisted surgical device 100 may provide various data, information, or images to, for example, the surgical robot 300 to assist robotic surgery. The robotically-assisted surgical device 100 may acquire, from, for example, the surgical robot 300, information regarding a mechanism or the operation of the surgical robot 300 or data obtained before, during, or after robotic surgery such that various kinds of analysis or interpretation can be performed based on the acquired information or data. The analysis result or the interpretation result may be visualized.

A measuring instrument 400 is connected to the robotically-assisted surgical device 100 through the communication unit 110. The measuring instrument 400 may measure information (for example, a body surface position of the subject PS) regarding the subject PS (for example, a patient) to be operated by the surgical robot 300. The measuring instrument 400 may measure a position of the port PT provided on the body surface of the subject PS. The measuring instrument 400 may be, for example, a depth sensor 410. The depth sensor 410 may be included in the surgical robot 300 (for example, the robot main body) or may be provided in the ceiling or the like of an operating room where robotic surgery is performed. In addition, the measuring instrument 400 may receive an input of the result of manual measurement of an operation unit of the measuring instrument 400. In the manual measurement, for example, information regarding a patient or a port position on the body surface may be measured by a ruler or a tape measure.

In addition, the CT apparatus 200 is connected to the robotically-assisted surgical device 100. Alternatively, instead of the CT apparatus 200, a device capable of capturing various images may be connected to the robotically-assisted surgical device 100. This device may be, for example, an angiographic device or an ultrasound device. This device may be used to check the internal state of the subject PS before and during robotic surgery.

The CT apparatus 200 irradiates an organism with X-rays and acquires images (CT images) using a difference in X-ray absorption depending on tissues. The subject PS may be, for example, a human body or an organism. The subject PS may not be a human body nor an organism. For example, the subject PS may be an animal or a phantom for surgical training.

A plurality of CT images may be acquired in a time series. The CT apparatus 200 generates volume data including information regarding any portion inside the organism. Here, any portion inside the organism may include various organs (for example, brain, heart, kidney, colon, intestine, lung, chest, lacteal gland, and prostate gland). By acquiring the CT image, it is possible to obtain a pixel value (CT value, voxel value) of each pixel (voxel) of the CT image. The CT apparatus 200 transmits the volume data as the CT image to the robotically-assisted surgical device 100 by wire or a wirelessly.

Specifically, the CT apparatus 200 includes a gantry (not illustrated) and a console (not illustrated). The gantry includes an X-ray generator (not illustrated) and an X-ray detector (not illustrated) and acquires images at a predetermined timing instructed by the console to detect an X-ray transmitted through the subject PS and to obtain X-ray detection data. The X-ray generator includes an X-ray tube (not illustrated). The console is connected to the robotically-assisted surgical device 100. The console acquires a plurality of X-ray detection data from the gantry and generates volume data based on the X-ray detection data. The console transmits the generated volume data to the robotically-assisted surgical device 100. The console may include an operation unit (not illustrated) for inputting patient information, scanning conditions regarding CT scanning, contrast enhancement conditions regarding contrast medium administration, and other information. This operation unit may include an input device such as a keyboard or a mouse.

The CT apparatus 200 continuously captures images to acquire a plurality of 3D volume data such that a moving image can also be generated. Data of the moving image generated the plurality of 3D volume data will also be referred to as 4D (four-dimensional) data.

The CT apparatus 200 may capture CT images at each of a plurality of timings. The CT apparatus 200 may capture a CT image in a state where the subject PS is contrast-enhanced. The CT apparatus 200 may capture a CT image in a state where the subject PS is not contrast-enhanced.

In the robotically-assisted surgical device 100, the communication unit 110 performs communication of various data or information with other devices. The communication unit 110 may perform communication of various data with the CT apparatus 200, the surgical robot 300, and the measuring instrument 400. The communication unit 110 performs wired communication or wireless communication. The communication unit 110 may be connected to the CT apparatus 200, the surgical robot 300, and the measuring instrument 400 in a wired or wireless manner.

The communication unit 110 may acquire various information for robotic surgery from the surgical robot 300. The various information may include, for example, kinematic information of the surgical robot 300. The communication unit 110 may transmit various information for robotic surgery to the surgical robot 300. The various information may include, for example, information (for example, an image or data) generated by a processing unit 160.

The communication unit 110 may acquire various information for robotic surgery from the measuring instrument 400. The various information may include, for example, position information of the body surface of the subject PS or information of a port position pierced on the body surface of the subject PS that is measured by the measuring instrument 400.

The communication unit 110 may acquire volume data from the CT apparatus 200. The acquired volume data may be transmitted immediately to the processor 140 for various processes, or may be stored in the memory 150 first and then transmitted to the processor 140 for various processes as necessary. In addition, the volume data may be acquired via a recording medium.

The volume data acquired by the CT apparatus 200 may be transmitted from the CT apparatus 200 to an image data server such as (PACS: Picture Archiving and Communication Systems; not illustrated) and stored therein. Instead of acquiring from the CT apparatus 200, the communication unit 110 may acquire volume data from the image data server. This way, the communication unit 110 functions as an acquisition unit that acquires various data such as volume data.

The UI 120 may include a touch panel, a pointing device, a keyboard, or a microphone. The UI 120 receives an input operation from a user of the robotically-assisted surgical device 100. The user may include a doctor, a radiographer, or other paramedic staffs. The doctor may include an operator that manipulates the robot operation terminal to operate robotic surgery or an assistant that assists robotic surgery near the subject PS.

The UI 120 receives an operation such as a designation of a region of interest (ROI), a setting of luminance conditions, and the like in the volume data. The region of interest may include various tissues (such as blood vessels, bronchial tubes, organs, bones, brain, heart, feet, neck, and blood flow). The tissues may broadly include tissues of the subject PS such as diseased tissue, normal tissue, organs, and parts. In addition, the UI 120 may receive an operation such as a designation of the region of interest, or a setting of luminance conditions in the volume data with respect to an image (for example, a 3D image or a 2D image described below) based on the volume data.

The display 130 may include a Liquid Crystal Display (LCD) and display various information. The various information may include a 3D image or a 2D image obtained from the volume data. The 3D image may include, for example, a volume rendering image, a surface rendering image, a virtual endoscope image (VE image), a virtual ultrasound image, or a Curved Planar Reconstruction (CPR) image. The volume rendering image may include a RaySum image (also simply referred to as “SUM image”), a Maximum Intensity Projection (MIP) image, a Minimum Intensity Projection (MinIP) image, an average image, or a Raycast image. The 2D image may include an axial image, a sagittal image, a coronal image, a Multi Planar Reconstruction (MPR) image, or the like. The 3D image and the 2D image may include a color fusion image.

The memory 150 may include a primary storage device such as various Read Only Memories (ROMs) or Random Access Memories (RAMs). The memory 150 may include a secondary storage device such as a Hard Disk Drive (HDD) or a Solid State Drive (SSD). The memory 150 may include a third storage device such as a USB memory or an SD card. The memory 150 stores various information. The various information includes information acquired via the communication unit 110, information and an image generated from the processor 140, setting information set by the processor 140, and various programs. The information acquired via the communication unit 110 may include, for example, information from the CT apparatus 200 (for example, volume data), information from the surgical robot 300, information from the measuring instrument 400, or information from an external server. The memory 150 is an example of a non-transitory recording medium in which a program is recorded.

A projection unit 170 projects visible light (for example, laser light) to the subject. The projection unit 170 projects the visible light to display various information (for example, the information of the port position) on the body surface of the subject PS (for example, the body surface of the abdomen). The visible light, that is, the information displayed on the body surface of the subject PS is recognized by the users (for example, an assistant).

The processor 140 may include a Central Processing Unit (CPU), a Digital Signal Processor (DSP), or a Graphical Processing Unit (GPU). The processor 140 executes the program stored in the memory 150 to function as the processing unit 160 controlling various processes and controls.

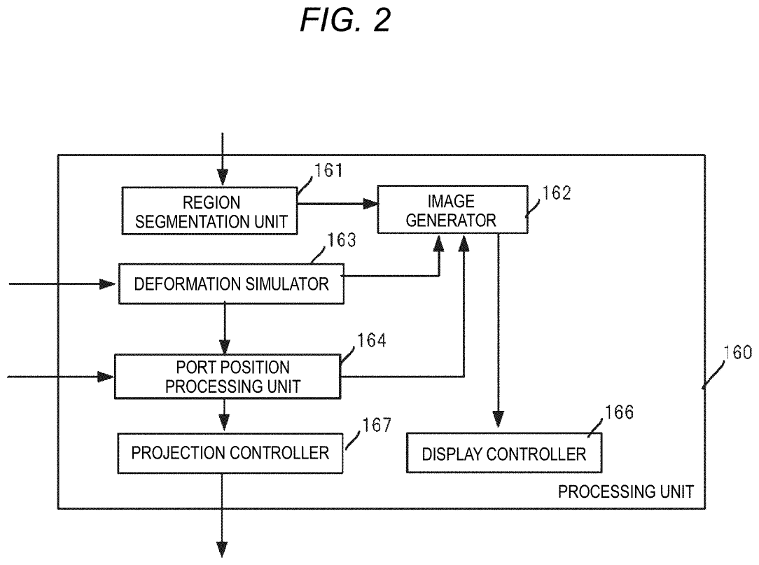

FIG. 2 is a block diagram illustrating a functional configuration example of the processing unit 160.

The processing unit 160 includes a region segmentation unit 161, an image generator 162, a deformation simulator 163, a port position processing unit 164, a display controller 166, and a projection controller 167.

The processing unit 160 integrates the respective units of the robotically-assisted surgical device 100. The respective sections included in the processing unit 160 may be implemented as different functions by one piece of hardware or may be implemented as different functions by a plurality of pieces of hardware. In addition, the respective sections included in the processing unit 160 may be implemented by a dedicated hardware component.

The region segmentation unit 161 may perform segmentation processing in the volume data. In this case, the UI 120 receives an instruction from a user and transmits information of the instruction to the region segmentation unit 161. The region segmentation unit 161 may perform segmentation processing from the volume data based on the information of the instruction using a well-known method to segment the region of interest. In addition, the region of interest may be set manually in accordance with the specific instruction from the user. In addition, when an observation target is predetermined, the region segmentation unit 161 may perform segmentation processing from the volume data to segment the region of interest including the observation target without the user instruction. The segmented region may include regions of various tissues (for example, blood vessels, bronchial tubes, organs, bones, brain, heart, feet, neck, blood flow, lacteal gland, chest, and tumor). The observation target may be a target to be treated by robotic surgery.

The image generator 162 may generate a 3D image or a 2D image based on the volume data acquired from the communication unit 110. The image generator 162 may generate a 3D image or a 2D image from the volume data acquired from the communication unit 110 based on a designated region or the region segmented by the region segmentation unit 161.

The deformation simulator 163 may perform a process relating to deformation in the subject PS as a surgery target. For example, the deformation simulator 163 may perform a pneumoperitoneum simulation of virtually performing pneumoperitoneum on the subject PS. A specific method of the pneumoperitoneum simulation may be a well-known method, for example, a method described in Takayuki Kitasaka, Kensaku Mori, Yuichiro Hayashi, Yasuhito Suenaga, Makoto Hashizume, and Junichiro Toriwaki, “Virtual Pneumoperitoneum for Generating Virtual Laparoscopic Views Based on Volumetric Deformation”, MICCAI (Medical Image Computing and Computer-Assisted Intervention), 2004, P559-P567 which is incorporated herein by reference.

That is, the deformation simulator 163 may perform the pneumoperitoneum simulation based on the volume data (volume data before pneumoperitoneum (non-pneumoperitoneum state)) acquired from the communication unit 110 or the region segmentation unit 161 to generate volume data after pneumoperitoneum (volume data in the pneumoperitoneum state). Through the pneumoperitoneum simulation, the user can simulate a state where pneumoperitoneum is performed on the subject PS without actually performing pneumoperitoneum on the subject PS to observe a state where pneumoperitoneum is virtually performed. Among pneumoperitoneum states, a state of pneumoperitoneum estimated by the pneumoperitoneum simulation will be referred to as “a virtual pneumoperitoneum state”, and a state where pneumoperitoneum is actually performed will also be referred to as “an actual pneumoperitoneum state”.

The pneumoperitoneum simulation may be a large deformation simulation using a finite element method. In this case, the deformation simulator 163 may segment a body surface including subcutaneous fat of the subject PS and an abdominal organ of the subject PS. The deformation simulator 163 may model the body surface as a two-layer finite element including skin and body fat and may model the abdominal organ as a finite element. The deformation simulator 163 may optionally segment a lung and a bone to be added to the model. The deformation simulator 163 may provide a gas region between the body surface and the abdominal organ and may extend (expand) the gas region (pneumoperitoneum space) by virtual gas injection.

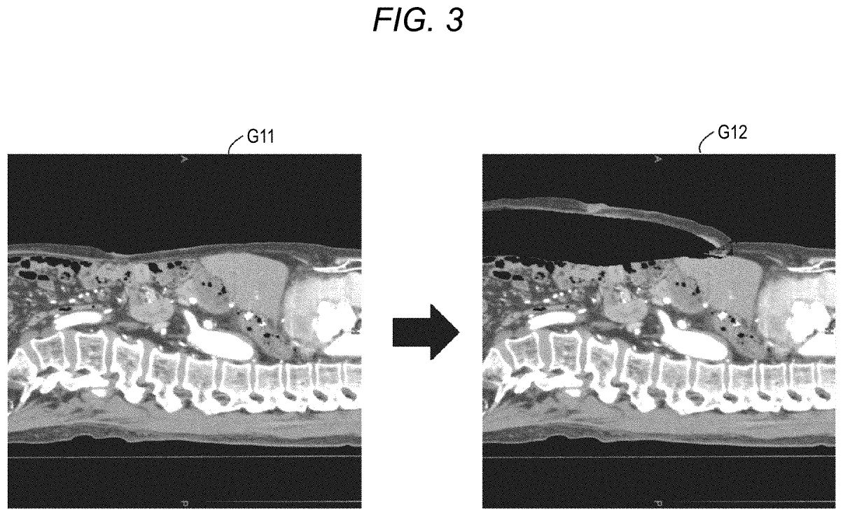

FIG. 3 is a view illustrating examples of MPR images of the abdomen before and after performing the pneumoperitoneum simulation. An image G11 illustrate the state before performing the pneumoperitoneum simulation, which is a state (non-pneumoperitoneum state) where the abdomen of the subject PS is not inflated. An image G12 illustrate the state after performing the pneumoperitoneum simulation, which is a state (virtual pneumoperitoneum state) where the abdomen of the subject PS is inflated and includes a pneumoperitoneum space KS. In robotic surgery, the subject PS is operated in the pneumoperitoneum state. Therefore, the pneumoperitoneum simulation may be performed on the volume data acquired in the non-pneumoperitoneum state by the deformation simulator 163 and the volume data in the virtual pneumoperitoneum state is derived.

The deformation simulator 163 may virtually deform the observation target such as an organ or a disease in the subject PS. The observation target may be a surgery target to be operated by the operator. The deformation simulator 163 may simulate a state where an organ is pulled, pressed, or dissected by the end effector EF. In addition, the deformation simulator 163 may simulate, for example, movement of an organ by a postural change.

The port position processing unit 164 acquires information of a plurality of ports PT provided on the body surface of the subject PS. The information of the port PT may include, for example, identification information of the port PT, information regarding a position (port position) on the body surface of the subject PS where the port PT is pierced, information regarding the size of the port PT, or the like. The information of a plurality of ports may be stored in the memory 150 or the external server as a template. The information of the plurality of ports may be determined according to the surgical procedure. The information of the plurality of ports may be used for preoperative planning.

The port position processing unit 164 may acquire the information of the plurality of ports positions from the memory 150. The port position processing unit 164 may acquire the information of the plurality of port positions from the external server via the communication unit 110. The port position processing unit 164 may receive a designation of port positions of the plurality of ports PT via the UI 120 to acquire the information of the plurality of port positions. The information of the plurality of ports may be the information of a combination of the plurality of port positions.

The port position processing unit 164 acquires kinematic information of the surgical robot 300. The kinematic information may be stored in the memory 150. The port position processing unit 164 may acquire the kinematic information from the memory 150. The port position processing unit 164 may acquire the kinematic information from the surgical robot 300 or the external server via the communication unit 110. The kinematic information may vary depending on the surgical robot 300.

The kinematic information may include, for example, shape information regarding the shape of an instrument (for example, the robot arm AR or the end effector EF) for robotic surgery included in the surgical robot 300 or operation information regarding the operation thereof. This shape information may include information of at least a part, for example, the length or weight of each portion of the robot arm AR or the end effector EF, the angle of the robot arm AR with respect to a reference direction (for example, a horizontal plane), or the inclination angle of the end effector EF with respect to the robot arm AR. This operation information may include information of at least a part, for example, the movable range of the robot arm AR or the end effector EF in the 3D space, the position, velocity, or acceleration of the robot arm AR during the operation of the robot arm AR, or the position, velocity, or acceleration of the end effector EF relative to the robot arm AR during the operation of the end effector EF.

In kinematics, not only the movable range of one robot arm but also the movable range of another robot arm are regulated. Accordingly, the surgical robot 300 operates based on the kinematics of each robot arm AR of the surgical robot 300, and therefore, interference between the plurality of robot arms AR during operation can be avoided.

The port position processing unit 164 acquires information of the surgical procedure. The surgical procedure refers to the procedure of surgery on the subject PS. The surgical procedure may be designated via the UI 120. Each treatment in robotic surgery may be determined depending on the surgical procedure. Depending on the treatment, the end effector EF required for the treatment may be determined. Accordingly, the end effector EF mounted on the robot arm AR may be determined depending on the surgical procedure, and the type of the end effector EF mounted on the robot arm AR may be determined depending on the surgical procedure. In addition, a minimum region that is required for the treatment or an effective region that is recommended to be secured for the treatment may be determined depending on the treatment.

The port position processing unit 164 acquires information of a target region. The target region may be a region including targets (for example, tissues (such as blood vessels, bronchial tubes, organs, bones, brain, heart, feet, and neck) to be treated by robotic surgery. The tissues may broadly include tissues of the subject PS such as diseased tissues, normal tissues, organs, and parts.

The port position processing unit 164 may acquire information regarding the position of the target region from the memory 150. The port position processing unit 164 may acquire the information of the position of the target region from the external server via the communication unit 110. The port position processing unit 164 may receive a designation of the position of the target region via the UI 120 to acquire the information regarding the position of the target region.

The port position processing unit 164 may execute a port position simulation. The port position simulation may be a simulation in which the user operates the UI 120 to determine whether or not desired robotic surgery can be performed on the subject PS. In the port position simulation, while simulating surgery, the user may operate the end effector EF inserted into each of the port positions in a virtual space to determine whether or not the target region as a surgery target is accessible. That is, in the port position simulation, while receiving the manual operation of the surgical robot 300, the user may determine whether or not a moving part (for example, the robot arm AR and the end effector EF) of the surgical robot 300 relating to robotic surgery is accessible to the target region as a surgery target without a problem. The port position processing unit 164 may obtain port position planning information through the port position simulation.

In the port position simulation, whether or not the target region is accessible may be determined based on the volume data of the subject PS, the acquired combination of the plurality of port positions, the kinematics of the surgical robot 300, the surgical procedure, the volume data of the virtual pneumoperitoneum state, and the like. While changing the plurality of port positions on the body surface of the subject PS, the port position processing unit 164 may determine whether or not the target region is accessible at each port position or may sequentially perform the port position simulation. The port position processing unit 164 may designate information regarding a finally preferable (for example, optimal) combination of port positions according to the user input via the UI 120. As a result, the port position processing unit 164 may plan the plurality of port positions to be pierced. The details of the port position simulation will be described below.

Using the plurality of port positions provided on the body surface of the subject PS, the port position processing unit 164 may derive (for example, calculate) a port position score representing the appropriateness for robotic surgery. That is, the port position score based on the combination of the plurality of port positions indicates the value of the combination of the plurality of port positions for robotic surgery. The port position score may be calculated based on the combination of the plurality of port positions, the kinematics of the surgical robot 300, the surgical procedure, the volume data of the virtual pneumoperitoneum state, and the like. The port position score is derived for each port position. The details of the port position score will be described below.

The port position processing unit 164 may adjust the port position based on the port position score. In this case, the port position processing unit 164 may adjust the port position based on the variation of the port position score according to the movement of the port position. The details of the port position adjustment will be described below.

As described above, the port position processing unit 164 may derive the plurality of port positions to be pierced according to the port position simulation. In addition, the port position processing unit 164 may derive the plurality of port positions to be pierced based on the port position score.

The display controller 166 causes the display 130 to display various data, information, or images. The display controller 166 may display the 3D image or the 2D image generated by the image generator 162. The display controller 166 may display an image showing the information of the plurality of ports PT (for example, the information of the port positions) generated by the image generator 162.

The projection controller 167 controls the projection of the visible light from the projection unit 170. The projection controller 167 may control, for example, a frequency or an intensity of the visible light, a position to which the visible light is projected, or a timing at which the visible light is projected.

The projection controller 167 causes the projection unit 170 to project the visible light to the subject PS and displays various information on the body surface of the subject PS (for example, the body surface of the abdomen). The projection controller 167 may project laser light to the body surface of the subject PS to mark a specific position on the body surface. The specific position may be, for example, the port position to be pierced or a position on the volume data where the observation target (for example, the affected part) is present when shifted from the specific position on the body surface in the normal direction. That is, the projection controller 167 may be a laser pointer indicating the port position.

In addition, the projection controller 167 may cause the projection unit 170 to project the visible light to the body surface of the subject PS to overlap and display information assisting robotic surgery (for example, the information regarding the port position) on the body surface of the subject PS. The overlapped information may be, for example, character information or graphic information. That is, the projection controller 167 may assist the user in robotic surgery using an augmented reality (AR) technique.

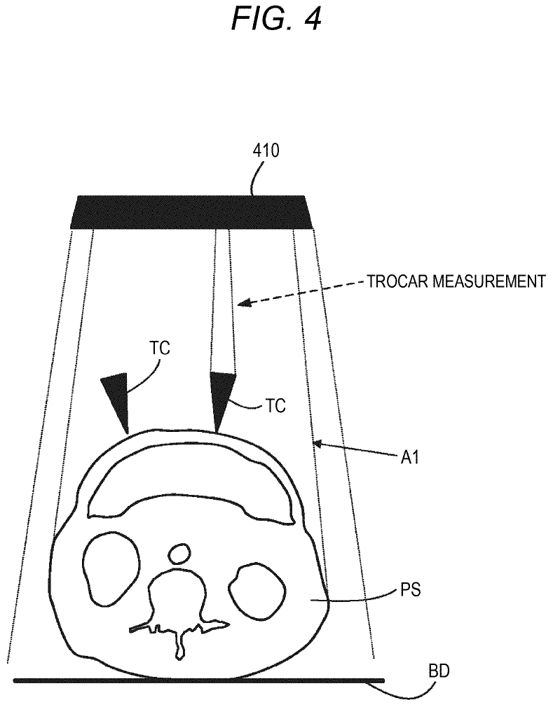

FIG. 4 is a view illustrating a measurement example of a port position of the pre-pierced port PT1. The measurement of the port position may be the 3D measurement. In FIG. 4, the subject PS (for example, a patient) is horizontally placed on a bed BD.

The depth sensor 410 may include: a light-emitting portion that emits infrared light; a light-receiving portion that receives infrared light, and a camera that captures an image. The depth sensor 410 may detect the distance from the depth sensor 410 to the subject PS based on the infrared light that is emitted from the light-emitting portion to the subject PS and reflected light that is reflected from the subject PS and received by the light-receiving portion. The depth sensor 410 may detect the upper, lower, left, and right sides of an object using the image captured by the camera. As a result, the depth sensor 410 may acquire information of a 3D position (3D coordinates) of each position (for example, the port position of the pre-pierced port PT1) on the body surface of the subject PS.

The depth sensor 410 may include a processor and an internal memory. The internal memory may store information regarding the shape of the trocar TC. Referring to the shape information of the trocar TC stored in the internal memory, the depth sensor 410 may detect (recognize) the trocar TC provided in the port PT pierced on the body surface of the subject PS to detect (measure) a 3D position of the trocar TC.

In addition, a predetermined mark may be formed on a surface of the trocar TC. The depth sensor 410 may capture an image using the predetermined mark on the trocar TC as a feature point to detect (recognize) the trocar TC by image recognition. As a result, the depth sensor 410 can improve the recognition accuracy of the trocar TC and can improve the measurement accuracy of the 3D position of the trocar TC.

In addition, the depth sensor 410 may include a stereo camera instead of the infrared sensor (the light-emitting portion and the light-receiving portion) such that the 3D position of the trocar TC can be measured by image processing. In this case, the depth sensor 410 may measure the 3D position of the trocar TC by recognizing the trocar TC by object recognition in an image captured by a stereo camera, detecting (recognizing) the position of the trocar TC on the body surface of the subject, and calculating the distance to the trocar TC.

The depth sensor 410 may measure each position or the position of the trocar TC on the body surface of the subject PS in a range that can be reached by the infrared light emitted from the infrared sensor or in a range where an image can be captured by the camera (refer to a range A1 in FIG. 4).

The deformation simulator 163 of the robotically-assisted surgical device 100 may acquire information regarding each position on the body surface of the subject PS in the actual pneumoperitoneum state, that is, information regarding the shape of the body surface of the subject PS in the actual pneumoperitoneum state from the depth sensor 410. In addition, the deformation simulator 163 may extract the contour (corresponding to the body surface) of the subject PS based on the volume data of the subject PS in the non-pneumoperitoneum state to acquire information regarding each position on the body surface of the subject PS in the non-pneumoperitoneum state, that is, information regarding the shape of the body surface of the subject PS in the non-pneumoperitoneum state.

The deformation simulator 163 may calculate a difference between each position on the body surface of the subject PS in the actual pneumoperitoneum state and each position on the body surface of the subject PS in the non-pneumoperitoneum state, that is, a difference between the shape of the body surface of the subject PS in the actual pneumoperitoneum state and the shape of the body surface of the subject PS in the non-pneumoperitoneum state. As a result, the deformation simulator 163 can recognize the amount of pneumoperitoneum for allowing the actual pneumoperitoneum state of the subject PS.

In addition, the deformation simulator 163 may correct a simulation method or a simulation result of the pneumoperitoneum simulation based on the difference between the actual pneumoperitoneum state and the virtual pneumoperitoneum state in the pneumoperitoneum simulation. That is, the deformation simulator 163 may correct a simulation method or a simulation result of the pneumoperitoneum simulation based on the actual amount of pneumoperitoneum. The deformation simulator 163 may store the correction information in the memory 150. In addition, the deformation simulator 163 may receive the amount of scavenging air from a pneumoperitoneum device via the communication unit 110 to correct a simulation method or a simulation result of the pneumoperitoneum simulation. As a result, the robotically-assisted surgical device 100 can improve the accuracy of the pneumoperitoneum simulation.

Next, an example of displaying a port position will be described.

The deformation simulator 163 performs the pneumoperitoneum simulation on the volume data obtained in the non-pneumoperitoneum state (for example, by preoperative CT scanning) to generate the volume data of the virtual pneumoperitoneum state. The image generator 162 may perform volume rendering on the volume data of the virtual pneumoperitoneum state to generate a volume rendering image. The image generator 162 may perform surface rendering on the volume data of the virtual pneumoperitoneum state to generate a surface rendering image.

The deformation simulator 163 may perform the pneumoperitoneum simulation on the volume data obtained in the non-pneumoperitoneum state (for example, by preoperative CT scanning) to generate deformation information regarding a destination (respective points after movement) to which respective points of the volume data of the non-pneumoperitoneum state are moved by pneumoperitoneum. The image generator 162 may apply the deformation information to the volume data obtained in the non-pneumoperitoneum state (for example, by preoperative CT scanning) to generate virtual pneumoperitoneum volume data. The image generator 162 may generate a surface from the virtual pneumoperitoneum volume data to generate a surface rendering image. The image generator 162 may generate a surface from the volume data acquired in the non-pneumoperitoneum state (for example, by preoperative CT scanning) to generate a surface rendering image. The image generator 162 may apply the deformation information to the surface generated from the volume data acquired in the non-pneumoperitoneum state (for example, by preoperative CT scanning) to generate a surface rendering image of the virtual pneumoperitoneum state. The deformation information may include information regarding at least the movement of a port position by pneumoperitoneum. The image generator 162 may segment bones from the volume data obtained in the non-pneumoperitoneum state (for example, by preoperative CT scanning) such that the bones are excluded from the deformation information as an element that is not moved by pneumoperitoneum and the movement of other tissues may be generated as deformation information.

The display controller 166 may cause the display 130 to display the port position derived from the port position processing unit 164 so as to overlap the volume rendering image or the surface rendering image of the virtual pneumoperitoneum state.

The projection controller 167 may project visible light to the port position on the body surface of the subject PS (for example, a patient) derived by the port position processing unit 164 to indicate the port position using the visible light and to visualize the port position. As a result, the user can perform a treatment such as piercing on the port position while checking the port position on the body surface of the subject PS.

The projection controller 167 may project visible light to the subject PS to display information regarding the port position on the body surface of the subject PS (for example, a patient) derived by the port position processing unit 164. In this case, the projection controller 167 may display the information regarding the port position (for example, the identification information of the port or an arrow indicating the port position) to overlap the subject PS using an AR technique. As a result, referring to guide information by the visible light, the user can perform a treatment such as piercing on the port position while checking the information regarding the port position on the body surface of the subject PS.

Here, the deformation information will be described in detail.

The deformation simulator 163 generates the virtual pneumoperitoneum volume data from the volume data of the non-pneumoperitoneum state using the deformation information. Visualization of the deformation information itself and a specific deformation method of the volume data using the deformation information are described in, for example, U.S. Pat. No. 8,311,300 and Japanese Patent No. 5408493 which are incorporated herein by reference. These methods are methods in an example for non-rigid registration but will also be referred to as the movement analysis (deformation analysis) and movement analysis information (deformation information).

The deformation simulator 163 may acquire, as the deformation information, information regarding the amount of movement or information regarding the velocity at a given point of the volume data. When the method described in US2014/0148816A which is incorporated herein by reference is applied, the deformation simulator 163 separates the volume data into a 2D lattice node (k, l), and 2D coordinates (x, y) in a phase node (k, l, t) of a phase t of the 2D lattice is obtained. In this case, based on a difference between a plurality of nodes (k, l, t) obtained by changing the value of the phase t, the information regarding the amount of movement at the lattice point of the node (k, l) may be calculated. In addition, the deformation simulator 163 may differentiate the information regarding the amount of movement with time to calculate the information regarding the velocity. The information regarding the amount of movement or the velocity may be expressed by a vector.

When the deformation simulator 163 interpolates the deformation information of the 2D lattice at each point of the entire volume data, the deformation information of each point of the volume data can be obtained. When the deformation information of a predetermined point is applied to each point of a region including an observation site, the deformation information of each point of the region including the observation site can be obtained.

In addition, when the method described in Japanese Patent No. $408493 is applied, the deformation simulator 163 may generate the deformation information based on volume data tk−1 and time information tk−1 thereof, and volume data tk and time information tk thereof among a plurality of volume data aligned in time series. As a result, the robotically-assisted surgical device 100 can obtain accurate deformation information in consideration of the movement of an organ caused by breathing or heartbeat. The deformation information may indicate information regarding a corresponding position on the plurality of volume data or a corresponding relationship of a corresponding object, or information regarding the process of a change in the movement of the position and the object. A pixel of each volume data is an index indicating a position at any time between time k−1 and time k.

The deformation simulator 163 is not limited to the method of US2014/0148816A and may perform deformation analysis using another well-known method. The robotically-assisted surgical device 100 performs deformation analysis on each point or the observation site using the deformation information, and the position where any position in the subject has moved before and after pneumoperitoneum can be grasped.

The deformation information is not particularly limited as long as it is the result of the deformation simulation and a destination to which at least one point is moved by pneumoperitoneum is visualized. In addition, the deformation information may be visualized directly or indirectly. For example, the deformation information may be visualized as a combination of a lattice before deformation and a lattice after deformation. For example, the deformation information may be visualized as a movement vector of at least one point.

The deformation information represents a corresponding relationship of respective points between the volume data of the non-pneumoperitoneum state and the volume data of the virtual pneumoperitoneum state, and a plurality of deformation information and a plurality of virtual pneumoperitoneum state may be present. A state where the amount of pneumoperitoneum is 0 may be represented by a non-pneumoperitoneum state 0, and a state where the amount of pneumoperitoneum is more than 0 may be represented by a virtual pneumoperitoneum state P (PA, PB, PC, or . . . ). In this case, a reference sign of the deformation information may be represented by a combination of O and P (PA, PB, PC, or . . . ). That is, the deformation information OP represents a corresponding relationship of respective points between the volume data of the non-pneumoperitoneum state O and the volume data of the virtual pneumoperitoneum state P. Deformation information OPA represents a corresponding relationship of respective points between the volume data of the non-pneumoperitoneum state O and volume data of the virtual pneumoperitoneum state PA in which the amount of pneumoperitoneum is A. Deformation information OPB represents a corresponding relationship of respective points between the volume data of the non-pneumoperitoneum state O and volume data of the virtual pneumoperitoneum state PB in which the amount of pneumoperitoneum is B.

A plurality of deformation information and a plurality of virtual pneumoperitoneum states may be present using different pneumoperitoneum conditions of the pneumoperitoneum simulation. The pneumoperitoneum condition may be a parameter indicating the amount of air supplied during pneumoperitoneum. The amount of air supplied may be, for example, the injection amount of gas, a gas pressure, or a gas volume in the abdominal cavity. The lung volume, the pulmonary function, the cardiac function, the age, the sex, the weight, the pre-existing diseases, or other factors may be used when a doctor determines the amount of air supplied during the pneumoperitoneum simulation. The pneumoperitoneum condition may include a parameter indicating a stretchability of a body tissue of the subject PS. For example, when the subject PS has experience of giving birth, the skin of the subject PS is likely to extend and is largely inflated even with the same amount of air supplied. The abdominal circumference, the subcutaneous fat, the surgical history, the age, the sex, the weight, the pre-existing diseases, or other parameters that affect the stretchability of a body tissue of the subject may be present. In addition, the easiness of extension of a body tissue of the subject PS may be a parameter based on which the hardness of an organ or a blood vessel can be estimated. In addition, the stretchability of a body tissue of the subject PS may vary depending on positions of the body surface. The pneumoperitoneum condition and the pneumoperitoneum simulation may be visualized with a large deformation simulation using a finite element method. The pneumoperitoneum condition and the pneumoperitoneum simulation may be visualized with a finite volume method, a level set approach, a lattice Boltzmann method, a CIP method (Constrained Interpolation Profile Scheme), or a combination thereof.

Next, a specific example of a standard port position will be described. The placement of the standard port position is also applicable to the embodiment.

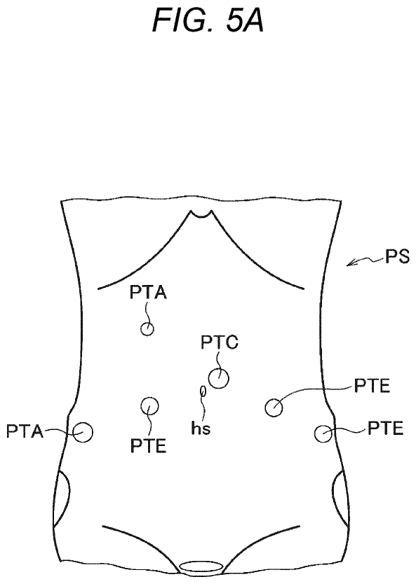

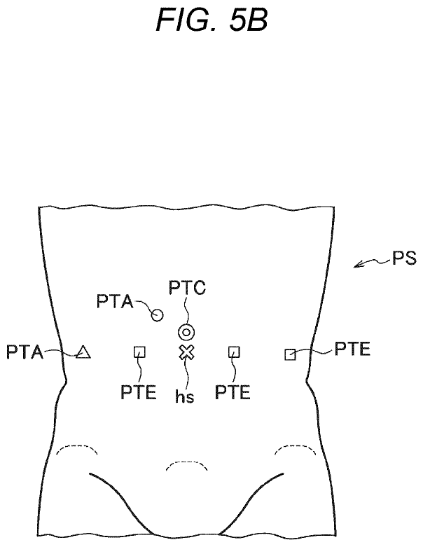

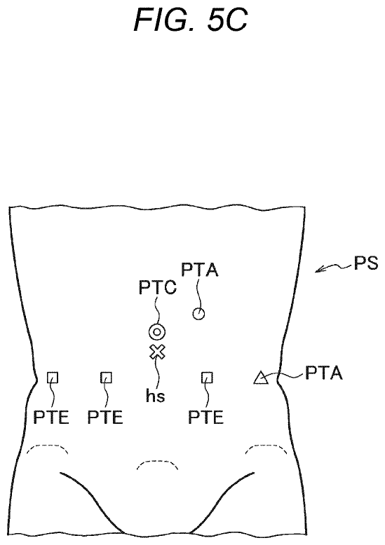

FIG. 5A is a view illustrating a first placement planning example of port positions placed on the body surface of the subject PS. FIG. 5B is a view illustrating a second placement planning example of port positions placed on the body surface of the subject PS. FIG. 5C is a view illustrating a third placement planning example of port positions placed on the body surface of the subject PS. The placement of a plurality of port positions may be planed, for example, according to the surgical procedure. In FIGS. 5A to 5C, the physical size of the subject PS or the position or size of a disease or the like of the observation target is not considered.

A plurality of port positions illustrated in FIGS. 5A to 5C are port positions that are planned to be pierced. There may be some errors between the port positions that are planned to be pierced and the port positions that are actually pierced. For example, there may be an error of about 25 mm.

The ports PT provided on the body surface of the subject PS may include a camera port PTC into which a camera CA is inserted, an end effector port PTE into which the end effector EF is inserted, and an auxiliary port PTA into which forceps held by an assistant are inserted. A plurality of ports PT may be present for each of the types (for example, for each of the camera port PTC, the end effector port PTE, and the auxiliary port PTA), or the sizes of the different types of ports PT may be the same as or different from each other. For example, the end effector port PTE into which the end effector EF for holding an organ or the end effector EF of which the movement in the subject PS is complex is inserted may be larger than the end effector port PTE into which the end effector EF as an electric knife is inserted. The placement position of the auxiliary port PTA may be planned relatively freely.

In FIG. 5A, large numbers of the end effector ports PTE and the auxiliary ports PTA are linearly arranged in the right direction of the subject PS and in the left direction of the subject PS, respectively, with respect to the port position of the camera port PTC as a reference (the vertex).

In FIG. 5B, large numbers of end effector ports PTE and the auxiliary ports PTA are linearly aligned with a position of a navel hs interposed therebetween. In addition, the camera port PTC is also placed near the navel hs.

In FIG. 5C, large numbers of end effector ports PTE and the auxiliary ports PTA are linearly aligned. The position of the navel hs is slightly shifted from the position on the straight line. In addition, the camera port PTC is also placed near the navel hs.

The reason why a large amount of ports PT are linearly placed in an existing plan is presumed to be that the user can easily position the port positions and feels safe. Among the plurality of ports PT, the camera port PTC may be placed at the center of the body surface of the subject PS.

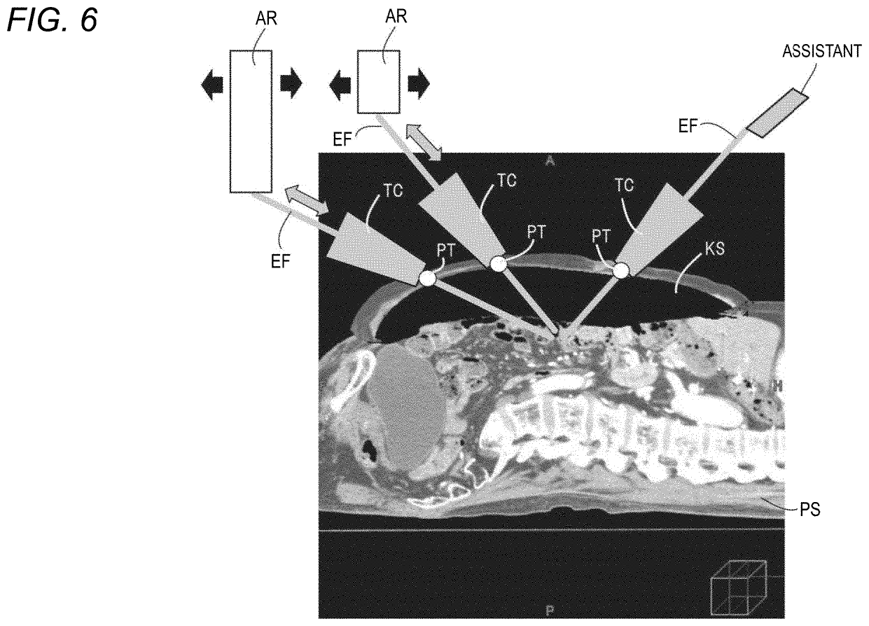

FIG. 6 is a view illustrating an example of a positional relationship between the subject PS, the ports PT, the trocars TC, and the robot arms AR during robotic surgery.

In the subject PS, one or more ports PT are provided. In each of the ports PT, the trocar TC is placed. The end effector EF is connected (for example, is inserted) to the trocar TC and a work (treatment) can be performed using the end effector EF in the subject. The port position is disposed to be fixed and does not move during operation. Accordingly, the position of the trocar TC disposed at the port position does not also move. On the other hand, according to the treatment during operation, the robot arms AR and the end effectors are controlled based on the manipulation of the robot operation terminal, and the robot arms AR move. Accordingly, the positional relationship between the robot arms AR and the trocars TC changes, and the angles of the trocars TC with respect to the body surface of the subject or the angles of the end effectors EF attached to the trocars TC change. In FIG. 6, a monitor held by an assistant is also illustrated as an end effector.

Next, the operation of the robotically-assisted surgical device 100 will be described.

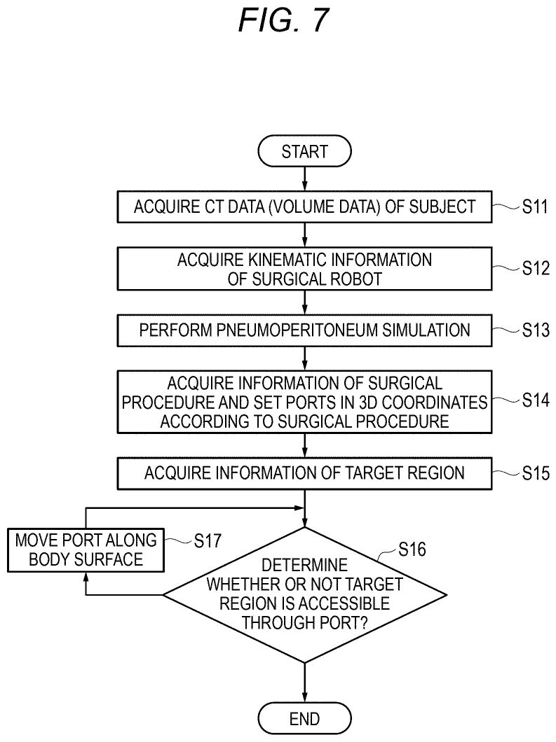

First, the procedure of the port position simulation will be described. FIG. 7 is a flowchart illustrating an example of the procedure of the port position simulation.

First, the port position processing unit 164 acquires the volume data including the subject PS, for example, via the communication unit 110 (S11). The port position processing unit 164 acquires the kinematic information from the surgical robot 300, for example, via the communication unit 110 (S12). The deformation simulator 163 performs the pneumoperitoneum simulation (S13) to generate the volume data of the virtual pneumoperitoneum state of the subject PS.

The port position processing unit 164 acquires the information of the surgical procedure (S14). The port position processing unit 164 acquires and sets the positions (initial positions) of the plurality of ports PT according to the acquired surgical procedure (S14). In this case, the port position processing unit 164 may set the positions of the plurality of ports PT in terms of 3D coordinates.

The port position processing unit 164 acquires the information of the target region (S15).

The port position processing unit 164 determines whether or not each of the end effectors EF inserted from each of the ports PT is accessible to the target region based on the positions of the plurality of ports acquired in S14 and the position of the target region (S16). Whether or not each of the end effectors EF is accessible to the target region may correspond to whether or not each of the end effectors EF can reach all the positions in the target region. That is, whether or not each of the end effectors EF is accessible to the target region shows that whether or not robotic surgery can be performed by the end effector EF (optionally, the plurality of end effectors EF) according to the acquired surgical procedure, and when each of the end effectors EF is accessible to the target region, robotic surgery can be performed.

When at least one of the end effectors EF is not accessible to at least a part of the target region, the port position processing unit 164 moves a port position of at least one port PT included in the plurality of ports PT to be pierced along the body surface of the subject PS (S17). In this case, the port position processing unit 164 may move the port position based on the user input via the UI 120. The port PT to be moved includes at least a port PT into which the end effector EF that is not accessible to at least a part of the target region is inserted.

When each of the end effectors EF is accessible to the target region, the processing unit 160 ends the process of the port position simulation of FIG. 7.

As described above, the robotically-assisted surgical device 100 performs the port position simulation such that whether or not each of the end effectors EF is accessible to the target region using the acquired plurality of port positions can be determined and thus whether or not robotic surgery can be performed by the surgical robot 300 using the acquired plurality of port positions can be determined. When the target region is not accessible using the plurality of port positions, at least a part of the port positions may be changed via the UI 120 to determine again whether or not the target region is accessible using the changed plurality of port positions. The robotically-assisted surgical device 100 can plan a combination of a plurality of port positions that are accessible to the target region as the plurality of port positions to be pierced. This way, the robotically-assisted surgical device 100 can plan the port position by the user manually adjusting the port position.

Next, an example of calculating the port position score will be described.

The plurality of port positions are determined, for example, according to the surgical procedure, and it may be assumed that each port position is disposed at any positions on the body surface of the subject PS. Accordingly, as the combination of the plurality of port positions, various combinations of port positions may be assumed. One end effector EF mounted on the robot arm AR can be inserted from one port PT into the subject PS. Accordingly, a plurality of end effectors EF mounted on a plurality of robot arms AR can be inserted from a plurality of ports PT into the subject PS.

A range where one end effector EF can reach the subject PS through the port PT is a working area (individual working area WA1) where a work (treatment in robotic surgery) can be performed by one end effector EF Accordingly, an area where the individual working areas WA1 of the plurality of end effectors EF overlap each other is a working area (entire working area WA2) where the plurality of end effectors EF can simultaneously reach the inside of the subject PS through the plurality of ports PT. In a treatment according to the surgical procedure, a predetermined number (for example, three) of end effectors EF needs to be operated at the same time. Therefore, the entire working area WA2 where the predetermined number of end effectors EF can simultaneously reach the inside of the subject PS is considered.

In addition, the position where the end effector EF can reach the subject PS varies depending on the kinematics of the surgical robot 300, and thus is added to the derivation of a port position as a position where the end effector EF is inserted into the subject PS. In addition, the position of the entire working area WA2 in the subject PS that is required to be secured varies depending on the surgical procedure, and thus is added to the derivation of a port position corresponding to the position of the entire working area WA2.

The port position processing unit 164 may calculate the port position score for each of the acquired (assumed) combinations of the plurality of port positions. The port position processing unit 164 may plan a combination of port positions having a port position score (for example, a maximum port score) that satisfies predetermined conditions among the assumed combinations of the plurality of port positions. That is, the plurality of port positions included in the planned combination of the port positions may be planned as the plurality of port positions to be pierced.

A relationship between the port position and the operation of the moving part of the surgical robot 300 may satisfy a relationship described in, for example, Mitsuhiro Hayashibe. Naoki Suzuki, Makoto Hashizume, Kozo Konishi, Asaki Hattori, “Robotic surgery setup simulation with the integration of inverse-kinematics computation and medical imaging”, computer methods and programs in biomedicine, 2006, P63-P72 and Pal Johan From, “On the Kinematics of Robotic-assisted Minimally Invasive Surgery”, Modeling Identification and Control, Vol. 34, No. 2, 2013, P69-P82, which are incorporated herein by reference.

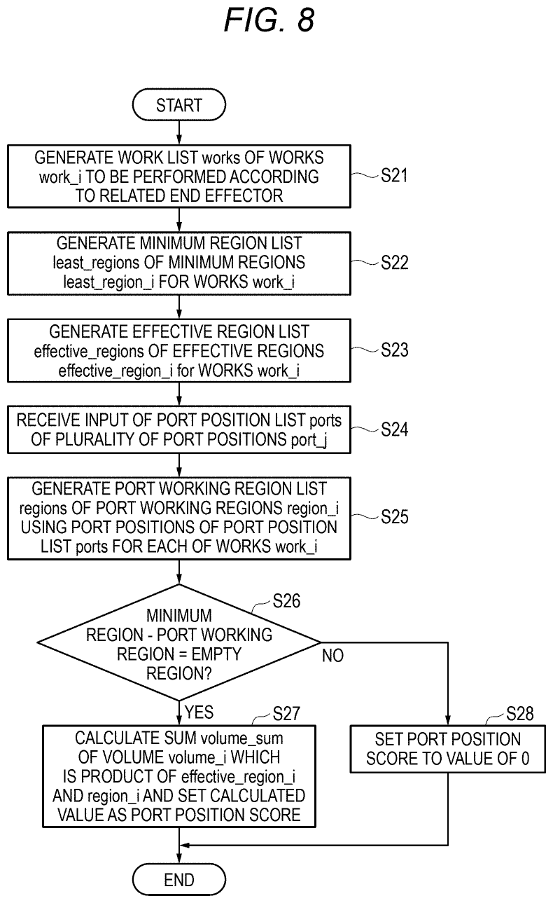

FIG. 8 is a flowchart illustrating an operation example when the port position score is calculated by the robotically-assisted surgical device 100.

Before the process of FIG. 8, the acquisition of the volume data of the subject PS, the acquisition of the kinematic information of the surgical robot 300, the execution of the pneumoperitoneum simulation, and the acquisition of the information of the surgical procedure are performed in advance as in S11 to S14 of the port position simulation illustrated in FIG. 7. In addition, the kinematic information may include the information of each of the end effectors EF mounted on each robot arm according to the surgical procedure. The initial value of the port position score is 0. The port position score is an evaluation function (evaluation value) indicating the value of the combination of the port positions. A variable is an example of identification information of a work, and a variable j is an example of identification information of a port.

The port position processing unit 164 generates a work list works, which is a list of works work_i in which each end effector EF is used, according to the surgical procedure (S21). The work work_i includes information for allowing each end effector EF to perform the work in the surgical procedure according to the surgical procedure. The work work_i may include, for example, gripping, dissection, or suture. The work may include a solo work that is performed by a single end effector EF or a cooperative work that is performed by a plurality of end effectors EF.

Based on the surgical procedure and the volume data of the virtual pneumoperitoneum state, the port position processing unit 164 plans a minimum region least_region_i, which is a region necessary for performing the works work_i included in the work list works (S22). The minimum region may be specified as a 3D region in the subject PS. The port position processing unit 164 generates a minimum region list Least_regions, which is a list of the minimum regions least_region_i (S22).

Based on the surgical procedure, the kinematics of the surgical robot 300, and the volume data of the virtual pneumoperitoneum state, the port position processing unit 164 plans an effective region effective_region_i, which is a region recommended for performing the work work_i included in the work list works (S23). The port position processing unit 164 generates an effective region list effective regions, which is a list of the effective regions effective_region_i (S23). The effective region may include not only the minimum space (minimum region) for performing the work but also a space that is effective, for example, the end effector EF to operate.

The port position processing unit 164 acquires information of a port position list ports, which is a list of a plurality of port positions port_j (S24). The port position may be specified by 3D coordinates (x, y, z). The port position processing unit 164 may receive, for example, a user input through the UI 120 to acquire the port position list ports including one or more port positions designated by the user. The port position processing unit 164 may acquire the port position list ports that is stored in the memory 150 as a template.

Based on the surgical procedure, the kinematics of the surgical robot 300, the volume data of the virtual pneumoperitoneum state, and the acquired plurality of port positions, the port position processing unit 164 plans a port working region region_i, which is a region where each of the end effectors EF can perform each of the works work_i through each of the port positions port_j (S25). The port working region may be specified as a 3D region. The port position processing unit 164 generates a port working region list regions, which is a list of the port working regions region_i (S25).

The port position processing unit 164 subtracts the port working region region_i from the minimum region least_region_i for each of the works work_i to calculate a subtracted region (subtracted value) (S26). The port position processing unit 164 determines whether or not the subtracted region is an empty region (the subtracted value is negative) (S26). Whether or not the subtracted region is an empty region shows that whether or not a region that is not covered with the port working region region_i (a region that cannot be reached by the end effector EF through the port PT) is present in at least a part of the minimum region least_region_i.

When the subtracted region is an empty region, the port position processing unit 164 calculates a volume value volume_i, which is the product of the effective region effective_region_i and the port working region region_i (S27). The port position processing unit 164 sums the volume values Volume_i calculated for each of the works work_i to calculate a sum value Volume_sum. The port position processing unit 164 sets the sum value Volume_sum as the port position score (S27).

That is, when the subtracted region is an empty region, it is preferable that the region that is not covered with the port working region is not present in the minimum region and this port position list ports (the combination of the port positions port_j) is selected. Therefore, in order to promote the selection of the port position list, the value for each of the works work_i is added to the port position score. In addition, by planning the port position score based on the volume value Volume_i, as the minimum region or the port working region increases, the port position score increases, and this port position list ports is more likely to be selected. Accordingly, the port position processing unit 164 is more likely to select a combination of port positions in which the minimum region or the port working region is large and each treatment is easy in surgery.

On the other hand, when the subtracted region is not an empty region, the port position processing unit 164 sets the port position score of the port position list ports to a value of 0 (S28). That is, since the region that is not covered with the port working region is present in at least a part of the minimum region and, the work of the target work work_i may not be completed, it is not preferable to select this port position list ports. Thus, in order to make the selection of the port position list ports difficult, the port position processing unit 164 sets the port position score to a value of 0 and excludes the port position list from candidates of the selection. In this case, when the subtracted region is an empty region in a case where another work work_i is performed using the same port position list ports, the port position processing unit 164 sets the port position score to a value of 0 as a whole.

The port position processing unit 164 may calculate a port position score for all the works work_i by repeating the respective steps of FIG. 8 for all the works work_i.

As described above, the robotically-assisted surgical device 100 derives the port position score, and when the robotic surgery is performed using the plurality of port positions provided on the body surface of the subject PS, the appropriateness of the combination of the port positions to be pierced can be grasped. The individual working area WA1 and the entire working area WA2 depend on the placement positions of the plurality of ports to be pierced. Even in this case, by using a score (port position score) for each combination of a plurality of port positions, the surgical robot 300 can derive a combination of a plurality of port positions in which, for example, the port position score is a threshold th1 or higher (for example, maximum), and the port positions with which robotic surgery can be easily performed can be set.

In addition, by appropriately securing the working area based on the port position score, the user can secure a wide visual field in the subject that cannot be directly visually observed in robotic surgery, a wide port working region can be secured, and unexpected events can be easily handled.

In addition, in robotic surgery, the port positions to be pierced are not variable. However, the robot arms AR on which the end effectors inserted into the port positions are mounted are movable in a predetermined range. Therefore, in robotic surgery, depending on the planned port positions, the robot arms AR may interfere with each other. Therefore, port position planning is important. In addition, the positional relationship between the surgical robot 300 and the subject PS cannot be changed during operation in principle. Therefore, port position planning is important.

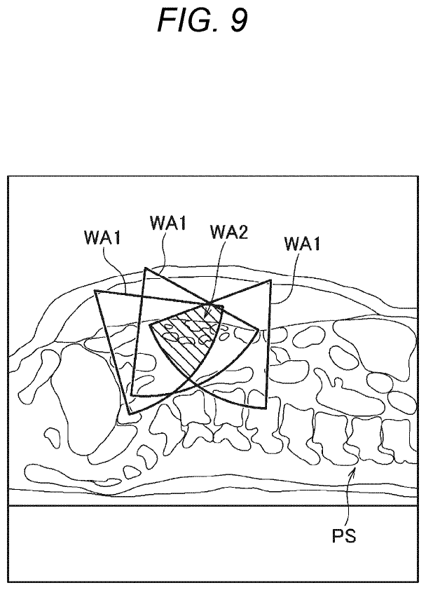

FIG. 9 is a view illustrating an example of working areas determined based on the port positions. The individual working area WA1 is an individual working area corresponding to each of the port positions port_j. The individual working area WA1 may be a region in the subject PS that can be reached by each of the end effectors. An area where the respective individual working areas WA1 overlap each other is the entire working area WA2. The entire working area WA2 may correspond to the port working region region_i. The robotically-assisted surgical device 100 can optimize each of the port positions using the port position score, and the suitable individual working areas WA1 and the suitable entire working area WA2 can be derived.

Next, the details of the port position adjustment will be described.

The port position processing unit 164 acquires information of the plurality of port positions (candidate positions), for example, based on the template stored in the memory 150 or the user instruction via UI 120. The port position processing unit 164 calculates the port position score for the case using the plurality of port positions based on the acquired combination of the plurality of port positions.

The port position processing unit 164 may adjust the position of the port PT based on the port position score. In this case, the port position processing unit 164 may adjust the position of the port PT based on the port position score for the acquired plurality of port positions and the port position score obtained when at least one port position among the plurality of port positions is changed. In this case, the port position processing unit 164 may also consider a small movement or a differential of the port position in each of the directions (x direction, y direction, and z direction) in a 3D space.

The x direction may be a direction along a left-right direction with respect to the subject PS. The y direction may be a forward-backward direction (thickness direction of the subject PS) with respect to the subject PS. The z direction may be an up-down direction (body axis direction of the subject PS) with respect to the subject PS. The x direction, the y direction, and the z direction may be three directions defined by Digital Imaging and COmmunications in Medicine (DICOM). The x direction, the y direction, and the z direction may be directions other than the above-described directions and are not necessarily the directions with respect to the subject PS.

For example, the port position processing unit 164 may calculate a port position score F (ports) for the plurality of port positions according to (Expression 1) to calculate a differential value F′ of F.

F(port_j(x+Δx,y,z))−F(port_j(x,y,z))

F(port_j(x,y+Δy,z))−F(port_j(x,y,z))

F(port_j(x,y,z+ΔA))−F(port_j(x,y,z)) (Expression 1)

That is, the port position processing unit 164 calculates the port position score F for the port position F (port_j(x+Δx, y, z)), calculates the port position score F for the port position F (port_j(x, y, z)), and calculates a difference therebetween. This difference value indicates a change in the port position score with respect to a small change of the port position F (port_j(x, y, z)) in the x direction, that is, the differential value F′ of F in the x direction.

In addition, the port position processing unit 164 calculates the port position score F for the port position F (port_j(x, y+Δy z)), calculates the port position score F for the port position F (port_j(x, y, z)), and calculates a difference therebetween. This difference value indicates a change in the port position score with respect to a small change of the port position F (port_j(x, y, z)) in the y direction, that is, the differential value F′ of F in the y direction.

In addition, the port position processing unit 164 calculates the port position score F for the port position F (port_j(x, y, z+Δz)), calculates the port position score F for the port position F (port_j(x, y, z)), and calculates a difference therebetween. This difference value indicates a change in the port position score with respect to a small change of the port position F (port_j(x, y, z)) in the z direction, that is, the differential value F′ of F in the z direction.

The port position processing unit 164 calculates a maximum value of the port position score based on the differential value F′ of each of the directions. In this case, the port position processing unit 164 may calculate a port position having the maximum port position score according to the steepest descent method based on the differential value F′. The port position processing unit 164 may adjust the port position to optimize the port position such that the calculated port position is a position to be pierced. Instead of the port position in which the port position score is the maximum, the port position may be, for example, a position in which the port position score is the threshold th1 or higher as long as the port position score is improved (increases).

The port position processing unit 164 may apply this port position adjustment to the adjustment of another port position included in the combination of the plurality of port positions or to the adjustment of port positions of another combination of a plurality of port positions. As a result, the port position processing unit 164 can plan the plurality of ports PT of which the respective port positions are adjusted (for example, optimized) as the port positions to be pierced. This way, the robotically-assisted surgical device 100 can plan the port position by automatically adjusting the port position.

Regarding the plurality of port positions (coordinates of the port positions), there may be an error of about a predetermined length (for example, 25 mm) between a piercing-planned position and an actual piercing position, and it is presumed that a port position planning accuracy of 3 mm at most is sufficient. Therefore, the port position processing unit 164 may set a plurality of port positions included in the combination of port positions as piercing-planned positions per predetermined length of the body surface of the subject PS, and the port position score may be calculated for each of the plurality of port positions. That is, the piercing-planned positions may be placed in a lattice shape (grid) of the predetermined length (for example, 3 mm) on the body surface of the subject PS. In addition, when it is assumed that the number of ports (for example, the number of intersections in a lattice shape) on the body surface is n and the number of ports included in the combination of port positions is m, the port position processing unit 164 may combine by sequentially selecting m port positions from n port positions and may calculate the port position score for each of the combinations. This way, when the grid is not excessively small as in a lattice shape having an interval of 3 mm, the calculation load of the port position processing unit 164 can be inhibited from being excessive, and the port position scores of all the combinations can be calculated.

The port position processing unit 164 may adjust the plurality of port positions using a well-known method. The port position processing unit 164 may plan the port positions to be pierced as the plurality of port positions included in the adjusted combination of port positions.

The well-known method of the port position adjustment may include techniques described in the followings: Shaun Selha, Pierre Dupont, Robert Howe, David Torchiana, “Dexterity optimization by port placement in robot-assisted minimally invasive surgery”, SPIE International Symposium on Intelligent Systems and Advanced Manufacturing, Newton, Mass., 28-31, 2001; Zhi Li, Dejan Milutinovic, Jacob Rosen, “Design of a Multi-Arm Surgical Robotic System for Dexterous Manipulation”, Journal of Mechanisms and Robotics, 2016; and US2007/0249911A, which are incorporated herein by reference.

Next, the movement of the port PT before and after pneumoperitoneum will be described.

In the pneumoperitoneum simulation, the type of a method of pneumoperitoneum may be used as a parameter. Examples of the method of pneumoperitoneum include an open method, a closed method, and a direct method.

In the open method, as in laparotomy, a first port is opened on the body surface of the subject PS. After verifying that the first port is safely pierced, a first trocar (Hasson Trocar) is inserted, and pneumoperitoneum is started. The open method has high safety, requires a long period of time, and is relatively invasive. Therefore, when adhesion is estimated to occur in the subject PS, the open method is performed.

In the closed method, a needle called a Veress needle (pneumoperitoneum needle) is pierced on the body surface of the subject PS to pierce the first port, gas is injected from the Veress needle to perform pneumoperitoneum on the subject PS, and the first trocar is inserted into the first port after pneumoperitoneum. In the closed method, a work is blindly performed, and thus the difficulty is high. The closed method is a rapid method, but an organ may be damaged.

In the direct method, the body surface of the subject PS is abruptly pierced using the first trocar for pneumoperitoneum. The direct method is a rapid method, but an organ may be damaged. There is a possibility that an organ is damaged, and the degree of the damage may increase in that case. However, unlike the closed method, a camera is attached to the first trocar for piercing. Therefore, a work is performed in a state where a visual field is secured.

In the open method and the direct method, the first port (usually, the camera port) into which the first trocar is inserted is pierced before pneumoperitoneum. A gas injection tube for pneumoperitoneum may be attached to the first trocar. Therefore, a special treatment may be required in the port position simulation and the port position adjustment. For example, the planning, placing, or marking port positions before pneumoperitoneum does not necessarily include the planning, placing, or marking of a port position of the first port into which the first trocar is inserted.

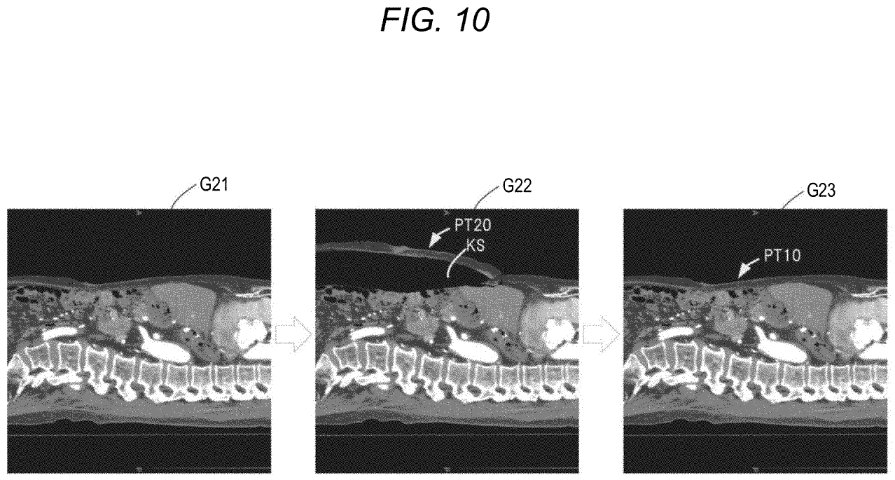

FIG. 10 is a view illustrating a movement example of a port position before and after the pneumoperitoneum simulation.

An image G21 is an MPR image of original CT data (volume data). That is, the image G21 is the MPR image of the volume data of the non-pneumoperitoneum state before performing the pneumoperitoneum simulation.

An image G22 is an MPR image of the volume data of the virtual pneumoperitoneum state where the pneumoperitoneum simulation is performed on the volume data of the image G21. In the image G22, a pneumoperitoneum space KS is present between an organ of the subject PS and the body surface. In the image G22, a position of a port PT20 planned in the volume data of the virtual pneumoperitoneum state is illustrated.

An image G23 is an MPR image of original CT data (volume data) as in the case of the image G21, which is an MPR image of the volume data of the non-pneumoperitoneum state. In the image G23, a position of a port PT10 in the volume data of the non-pneumoperitoneum state corresponding to the position of the port PT20 in the volume data of the virtual pneumoperitoneum state is illustrated. That is, the port PT10 on the body surface of the subject PS in the non-pneumoperitoneum state becomes the port PT20 on the body surface of the subject PS in the virtual pneumoperitoneum state. In addition, the port PT10 is expected to become the port PT20 even on the body surface of the subject PS in the actual pneumoperitoneum state. The positions of each of the ports PT10 and PT20 before and after the pneumoperitoneum simulation can be associated with each other by the above-described deformation information (movement information).

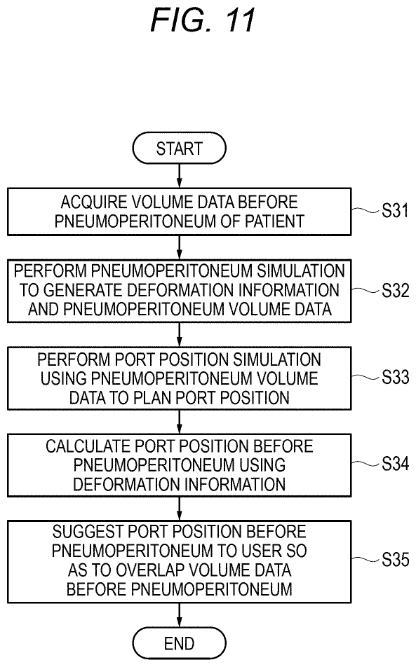

FIG. 11 is a flowchart illustrating an operation example when the port position before pneumoperitoneum is derived by the robotically-assisted surgical device 10. Before the process of FIG. 11, the acquisition of the kinematic information of the surgical robot 300 and the acquisition of the surgical procedure are performed in advance.

The deformation simulator 163 acquires the volume data of the subject PS (for example, a patient) via the communication unit 110 (S31). This volume data is the volume data of the non-pneumoperitoneum state (the volume data before pneumoperitoneum).

The deformation simulator 163 performs the pneumoperitoneum simulation on the volume data of the non-pneumoperitoneum state to generate the deformation information OP (S32). The deformation simulator 163 generates the volume data of the virtual pneumoperitoneum state based on the volume data of the non-pneumoperitoneum state and the deformation information (S32).

The port position processing unit 164 performs the port position simulation or the port position adjustment using the volume data of the virtual pneumoperitoneum state to plan the position (port position after pneumoperitoneum) of the port PT20 to be pierced on the body surface of the subject PS (S33).

The port position processing unit 164 calculates the position (port position before pneumoperitoneum) of the port PT10 corresponding to the position of the port PT20 in the volume data of the non-pneumoperitoneum state (before pneumoperitoneum) based on the position of the port PT20 planned based on the volume data of the virtual pneumoperitoneum state and the deformation information OP (S34).

The display controller 166 causes the display 130 to display, as the port position planned in the non-pneumoperitoneum state, information displaying the position (port position before pneumoperitoneum) of the port PT10 in the volume data of the non-pneumoperitoneum state so as to overlap the volume data of the non-pneumoperitoneum state (the rendering image based on the volume data) (S35).

As described above, by performing the operation of FIG. 11, the robotically-assisted surgical device 100 can derive the port position before pneumoperitoneum (the position of the port PT10) corresponding to the port position after pneumoperitoneum while taking the position of the port PT20 (port position after pneumoperitoneum) suitable for the state after pneumoperitoneum into consideration before performing pneumoperitoneum on the subject PS. In this case, the robotically-assisted surgical device 100 can derive the port position before pneumoperitoneum through simple arithmetic processing using the deformation information OP. In addition, by performing the planning, placing, marking, or the like of the port position before pneumoperitoneum, the robotically-assisted surgical device 100 can reduce the operative duration and a mental burden on the operator. When there is no error in the pneumoperitoneum simulation after pneumoperitoneum, that is, when there is no difference between the virtual pneumoperitoneum state and the actual pneumoperitoneum state, the marked port position before pneumoperitoneum matches the port position after actual pneumoperitoneum.

Next, the error of the pneumoperitoneum simulation will be described.

The error of the pneumoperitoneum simulation is generated by various factors. Here, errors generated by a variation in the stretchability of a body tissue of the subject PS depending on the subject PS will be mainly described. In particular, expansion caused by the pneumoperitoneum of the subject PS is not uniform on the body surface, and the error may be generated by this non-uniform expansion. In the pneumoperitoneum simulation, the stretchability of a body tissue is set at each position on the body surface. When this setting is not accurate, an error is generated.

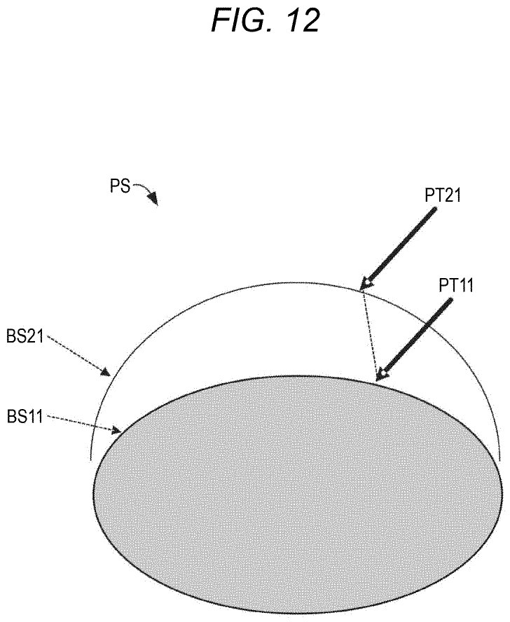

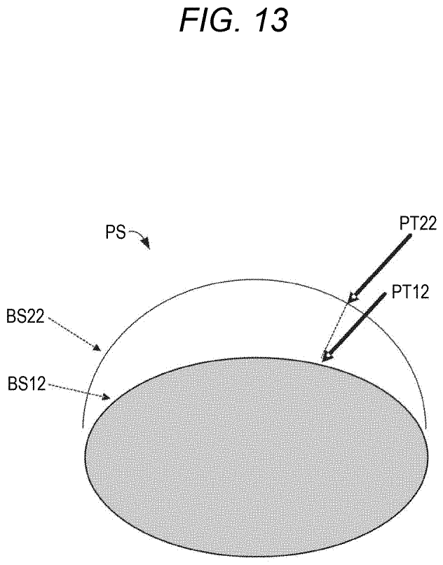

FIGS. 12 and 13 are views illustrating the handling of the non-uniform expansion of the subject PS in the pneumoperitoneum simulation.

During actual pneumoperitoneum, the body surface may non-uniformly expand. In other words, unlike an ideal balloon, a body surface BS1 does not uniformly expand during pneumoperitoneum. It can be said that the skin of the subject PS does not uniformly expand during actual pneumoperitoneum. That is, Young's modulus or an equivalent coefficient of the body surface of the subject PS varies depending on positions on the body surface BS1. In addition, the way of expansion during actual pneumoperitoneum may vary depending on the subject PS. Therefore, using different conditions (stretchability), a corresponding relationship between respective point on the body surface before actual pneumoperitoneum and respective points on the body surface after actual pneumoperitoneum is not uniquely determined.

Accordingly, when a plurality of conditions for Young's modulus or an equivalent coefficient of a body tissue on the body surface BS1 are prepared and the port position before pneumoperitoneum is derived from the port position after pneumoperitoneum based on the deformation information OP, the port position after pneumoperitoneum derived in the pneumoperitoneum simulation may vary depending on the conditions. Accordingly, when the pneumoperitoneum is performed on the port position before pneumoperitoneum, the port position before pneumoperitoneum corresponding to the port position after pneumoperitoneum may also not become a port position to be pierced after actual pneumoperitoneum. On the other hand, it can be expected that, when the robotically-assisted surgical device 100 derives a certain range as the port position after pneumoperitoneum or the port position before pneumoperitoneum derived in the pneumoperitoneum simulation in consideration of the non-uniform expansion on the body surface, misplacement from the port position after actual pneumoperitoneum can be suppressed.

Therefore, the deformation simulator 163 may perform a plurality of pneumoperitoneum simulations corresponding to the conditions for the stretchability of a body tissue on the respective positions on the body surface to perform the port position simulation or the port position adjustment based on the results of the respective pneumoperitoneum simulations. That is, the deformation simulator 163 may perform a plurality of pneumoperitoneum simulations using the plurality of different conditions. In this case, the port position processing unit 164 may calculate a plurality of port positions before pneumoperitoneum corresponding to the port positions after pneumoperitoneum determined by the port position simulation or the port position adjustment. In addition, the port position processing unit 164 may derive a certain range (port position range before pneumoperitoneum) as the port positions before pneumoperitoneum corresponding to the port positions after pneumoperitoneum determined by the port position simulation or the port position adjustment. Therefore, instead of calculating one port position before pneumoperitoneum, the port position processing unit 164 may calculate a range of port positions before pneumoperitoneum on the body surface BS1 or a plurality of points (positions) included in the range of port positions before pneumoperitoneum. Further, the deformation simulator 163 may perform a plurality of pneumoperitoneum simulations corresponding to the conditions for the stretchability of a body tissue on the respective positions in an internal organ to perform the port position simulation or the port position adjustment based on the results of the respective pneumoperitoneum simulations.

For each subject PS, the robotically-assisted surgical device 100 may store data representing the stretchability of a body tissue of the subject PS (for example, data representing the stretchability at each of body surface positions on the subject PS) in the memory 150 as an extension parameter indicating the way of extension of the body surface. The extension parameter indicates the way of extension of the body surface of the subject PS. The memory 150 may store a plurality of patterns of the extension parameter. As a result, the robotically-assisted surgical device 100 can take a variation in the stretchability of the body surface into consideration during the pneumoperitoneum simulation.

The deformation simulator 163 may add the extension parameter to parameters used for the pneumoperitoneum simulation to perform the pneumoperitoneum simulation. The deformation simulator 163 may perform a plurality of pneumoperitoneum simulations using different extension parameters to generate a plurality of volume data of the virtual pneumoperitoneum state. The port position processing unit 164 may derive a plurality of port positions after pneumoperitoneum based on the plurality of volume data of the virtual pneumoperitoneum state. The port position processing unit 164 may also derive port positions before pneumoperitoneum corresponding to the plurality of port positions after pneumoperitoneum. The port position processing unit 164 may derive a certain range (a range of port positions before pneumoperitoneum) including the derived plurality of port positions before pneumoperitoneum. In addition, the port position processing unit 164 may plan at least one point included in the range of port positions before pneumoperitoneum as the port position before pneumoperitoneum.

In FIGS. 12 and 13, as the port positions after pneumoperitoneum, positions of ports PT21 and PT22 on body surfaces BS21 and BS22 after pneumoperitoneum are obtained. As the port positions before pneumoperitoneum, positions of ports PT11 and PT12 on body surfaces BS11 and BS12 before pneumoperitoneum are obtained. The port positions after pneumoperitoneum (ports PT21 and PT22) can be derived by the port position simulation or the port position adjustment based on the volume data of the virtual pneumoperitoneum state to which the extension parameter is added. The port positions before pneumoperitoneum can be derived based on the port positions after pneumoperitoneum and the deformation information OP.

In FIGS. 12 and 13, for simplicity, the shape of the body surface BS21 after pneumoperitoneum of the subject PS under a condition COND1 is the same as the shape of the body surface BS22 after pneumoperitoneum of the subject PS under a condition COND2. The position (port position before pneumoperitoneum) of the port. PT11 on the body surface BS11 before pneumoperitoneum of the subject PS under the condition COND1 is the same as the position (port position before pneumoperitoneum) of the port PT12 on the body surface BS12 before pneumoperitoneum of the subject PS under the condition COND2. On the other hand, the position (port position after pneumoperitoneum) of the port PT21 on the body surface BS21 after pneumoperitoneum of the subject PS under the condition COND1 is different from the position (port position after pneumoperitoneum) of the port PT22 on the body surface BS22 after pneumoperitoneum of the subject PS under the condition COND2.

This way, as illustrated in FIGS. 12 and 13, even when the shapes of the body surfaces after pneumoperitoneum are the same, port positions after pneumoperitoneum corresponding to the same port position before pneumoperitoneum may be different from each other. Conversely, port positions before pneumoperitoneum corresponding different port positions after pneumoperitoneum may also be the same. Even when the body surfaces BS21 and BS22 after pneumoperitoneum are the same, the port position processing unit 164 can derive different port positions before pneumoperitoneum on the body surfaces BS11 and BS12 corresponding to the port positions after pneumoperitoneum through the different pneumoperitoneum simulations to which the extension parameter is added. The port position processing unit 164 can derive a range of port positions including these different port positions before pneumoperitoneum. In addition, the port position processing unit 164 can perform the port position simulation or the port position adjustment in consideration of a difference between the port positions after pneumoperitoneum (ports PT21 and PT22) depending on the conditions.

The image generator 162 renders the volume data of the non-pneumoperitoneum state to generate a rendering image. The display controller 166 may display information indicating the derived plurality of port positions before pneumoperitoneum or the derived range of port positions before pneumoperitoneum on the body surfaces BS11 and BS12 of the non-pneumoperitoneum state together with the rendering image of the non-pneumoperitoneum state. In addition, the display controller 166 may display the information indicating the derived plurality of port positions before pneumoperitoneum or the derived range of port positions before pneumoperitoneum on the body surfaces BS11 and BS12 of the non-pneumoperitoneum state together with surface rendering images representing the body surfaces BS11 and BS13 of the non-pneumoperitoneum state instead of the rendering image of the non-pneumoperitoneum state.

This way, the robotically-assisted surgical device 100 performs a plurality of pneumoperitoneum simulations in consideration of the non-uniform extension depending on the positions on the body surface of the subject PS such that one or more port position before pneumoperitoneum or a range of port positions before pneumoperitoneum can be derived. Accordingly, the robotically-assisted surgical device 100 can plan the port positions or the range of port positions before pneumoperitoneum. The user can perform the placement of the port positions or the piercing of the ports while checking the port positions before pneumoperitoneum or the range of port positions before pneumoperitoneum in consideration of the non-uniform extension of the subject PS. Accordingly, even when the extension of the subject PS in the pneumoperitoneum simulation is different from the actual extension of the subject PS, the robotically-assisted surgical device 100 can suppress the port positions from being misplaced at unexpected positions after pneumoperitoneum during operation.

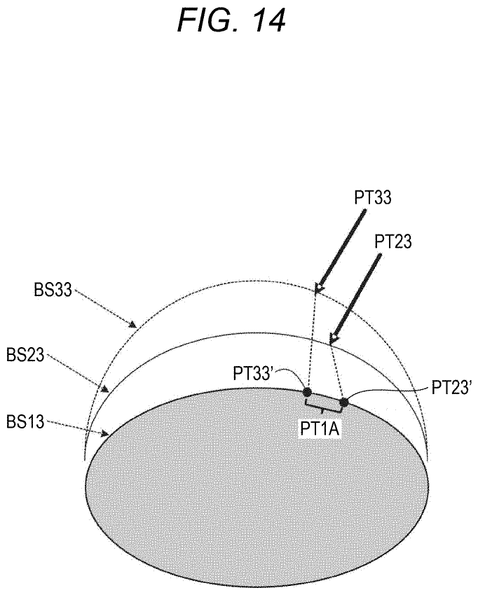

FIG. 14 is a view illustrating the handling of errors generated by a change in the amount of gas in the pneumoperitoneum simulation depending on circumstances during operation.

In the pneumoperitoneum simulation, the way that the body surface of the subject PS inflates varies depending on the injection amount of gas (for example, CO₂ gas) used for pneumoperitoneum. The injection amount of CO₂ gas depends on the pulmonary function of the subject PS (for example, a patient). In addition, the injection amount of CO₂ gas is under the control of an anesthetist. Therefore, the injection amount of CO₂ gas planned before the operation may be different from the injection amount of CO₂ gas used during the operation. Depending on the injection amount of CO₂ gas, the amount of pneumoperitoneum is determined.

Therefore, the deformation simulator 163 may perform a plurality of pneumoperitoneum simulations corresponding to the injection amounts of CO_{z} gas to perform the port position simulation or the port position adjustment based on the results of the respective pneumoperitoneum simulations. That is, the deformation simulator 163 may perform a plurality of pneumoperitoneum simulations using the plurality of different amounts of pneumoperitoneum. In this case, the port position processing unit 164 may calculate a plurality of port positions before pneumoperitoneum corresponding to the port positions after pneumoperitoneum determined by the port position simulation or the port position adjustment. In addition, the port position processing unit 164 may derive a certain range (a range of port positions before pneumoperitoneum) as the port positions before pneumoperitoneum corresponding to the port positions after pneumoperitoneum determined by the port position simulation or the port position adjustment. Therefore, instead of calculating one port position before pneumoperitoneum, the port position processing unit 164 may calculate a range of port positions before pneumoperitoneum on the body surface BS1 or a plurality of points (positions) included in the range of port positions before pneumoperitoneum.

In FIG. 14, a body surface BS13 of the non-pneumoperitoneum state and body surfaces BS23 and BS33 of the subject PS of a plurality of virtual pneumoperitoneum states PA and PB where pneumoperitoneum is performed in a plurality of different amounts of pneumoperitoneum A and B, respectively, are illustrated. The port position processing unit 164 performs the port position simulation or the port position adjustment based on volume data of the virtual pneumoperitoneum state PA for the body surface BS23 to derive a position of a port PT23 on the body surface BS23. The port position processing unit 164 performs the port position simulation or the port position adjustment based on volume data of the virtual pneumoperitoneum state PB for the body surface BS33 to derive a position of a port PT33 on the body surface BS33.

In addition, when the port position processing unit 164 derives a port position on the body surface BS13 corresponding to the position of the port PT23 based on the deformation information OPA indicating the corresponding relationship of the respective points between the volume data of the non-pneumoperitoneum state O and the volume data of the virtual pneumoperitoneum state PA, the port position becomes a position of a port PT23′. When the port position processing unit 164 derives a port position on the body surface BS13 corresponding to the position of the port PT33 based on the deformation information OPB indicating the corresponding relationship of the respective points between the volume data of the non-pneumoperitoneum state O and the volume data of the virtual pneumoperitoneum state PB, the port position becomes a position of a port PT33′.

Herein, when the amount of pneumoperitoneum is between the amount of pneumoperitoneum A for the body surface BS23 and the amount of pneumoperitoneum for the body surface BS33, the port position before pneumoperitoneum can be estimated to be present between the position of the port PT23′ and the position of the port PT33′. Accordingly, the port position processing unit 164 may derive a port position range PT1A before pneumoperitoneum as the port position before pneumoperitoneum in consideration of a plurality of amounts of pneumoperitoneum, that is, in consideration of the error in the amount of pneumoperitoneum (amount of gas) between the virtual pneumoperitoneum and the actual pneumoperitoneum. The port position range PT1A before pneumoperitoneum includes a range between the position of the port PT23′ and the position of the port PT33′.

The image generator 162 renders the volume data of the non-pneumoperitoneum state to generate a rendering image. The display controller 166 may display information indicating the port position range PT1A before pneumoperitoneum on the body surface BS13 of the non-pneumoperitoneum state together with the rendering image of the non-pneumoperitoneum state. In addition, the display controller 166 may display the information indicating the port position range PT1A before pneumoperitoneum together with a surface rendering image representing the body surface BS13 of the non-pneumoperitoneum state instead of the rendering image of the non-pneumoperitoneum state. In addition, the display controller 166 may simply display information indicating the position of the port PT23′ or the port PT33′ instead of the port position range PT1A before pneumoperitoneum. As a result, the user can check the port position range PT1A before pneumoperitoneum in consideration of the port positions of the body surfaces BS23 and BS33 on the body surface BS13.

This way, in consideration of the error corresponding to the amount of gas in the pneumoperitoneum simulation, the robotically-assisted surgical device 100 can estimate port positions before pneumoperitoneum corresponding to the respective port positions after pneumoperitoneum, for example, by assuming a range of the amount of pneumoperitoneum assumed as the amount of pneumoperitoneum, assuming a body surface for the maximum amount of pneumoperitoneum and a body surface for the minimum amount of pneumoperitoneum, and therefore, can estimate the port position range before pneumoperitoneum. As a result, even when the amount of pneumoperitoneum changes to some extent during the operation, the robotically-assisted surgical device 100 can suppress the port positions from being misplaced at unexpected positions after pneumoperitoneum during the operation. In addition, in the robotically-assisted surgical device 100, by assuming the amount of pneumoperitoneum to have a certain range from the beginning, the influence of the variation in the amount of pneumoperitoneum on the derivation of the port positions before pneumoperitoneum can be absorbed within an error range.

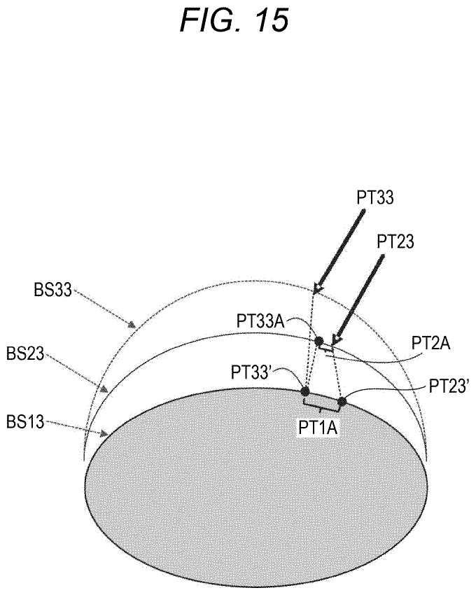

FIG. 15 is a view illustrating a display example relating to the error corresponding to the amount of gas in the pneumoperitoneum simulation. FIG. 15 illustrates a corresponding positional relationship on body surfaces of different virtual pneumoperitoneum states. In the description with reference to FIG. 15, the detailed description of the same features as those in FIG. 14 will not be repeated or will be simplified.

In FIG. 15, as in FIG. 14, the position of the port PT23′ on the body surface 13 corresponding to the position of the port PT23 on the body surface BS23 is derived. In addition, the position of the port PT33′ on the body surface 13 corresponding to the position of the port PT33 on the body surface BS33 is derived.

The port position processing unit 164 calculates a position of a port PT33A on the body surface BS23 corresponding to the position of the port PT33′ on the body surface BS13 based on the deformation information OPA. That is, it can be estimated that the position of the port PT33′ on the body surface BS33 of the amount of pneumoperitoneum B corresponds to the position of the port PT33A on the body surface BS23 of the amount of pneumoperitoneum A. Accordingly, the port position processing unit 164 can obtain a port position range PT2A, which is a range between the port PT23 and the port PT33A on the body surface BS23 of the amount of pneumoperitoneum B, through the port position simulation or the port position adjustment using the volume data of the amount of pneumoperitoneum A and the port position simulation or the port position adjustment using the volume data of the amount of pneumoperitoneum B. The port position range PT2A of the body surface BS23 corresponds to the port position range PT1A before pneumoperitoneum on the body surface BS13.

The image generator 162 may render the volume data of the amount of pneumoperitoneum B to generate a rendering image. The display controller 166 may display information indicating the port position range PT2A on the body surface BS23 with the amount of pneumoperitoneum B together with the rendering image of the amount of pneumoperitoneum B. In addition, the display controller 166 may display the information indicating the port position range PT2A together with a surface rendering image representing the body surface BS23 of the amount of pneumoperitoneum B instead of the rendering image of the amount of pneumoperitoneum B. In addition, the display controller 166 may simply display information indicating the position of the port PT33A instead of the port position range PT2A. As a result, the user can check the port position range PT2A in consideration of the port positions of the body surface BS33 on the body surface BS23.

In addition, the port position simulation or the port position adjustment may be performed assuming only one amount of pneumoperitoneum. Even in this case, the robotically-assisted surgical device 100 can derive a port PT 33B on the body surface BS23 of the amount of pneumoperitoneum A corresponding to the port PT33 on the body surface BS33 of the amount of pneumoperitoneum B based on the deformation information OPB and the deformation information OPA. For example, even when the amount of pneumoperitoneum assumed in the preoperative simulation (for example, the port position simulation or the port position adjustment) is different from the actual amount of pneumoperitoneum, the robotically-assisted surgical device 100 can derive the port position before pneumoperitoneum or the port position range before pneumoperitoneum by assuming the range of the amount of pneumoperitoneum as the actual amount of pneumoperitoneum in advance. Accordingly, the robotically-assisted surgical device 100 can estimate the corresponding relationship of the port positions between the respective pneumoperitoneum states in consideration of a plurality of virtual pneumoperitoneum states while reducing the number of times for the pneumoperitoneum simulation.

Here, the image generator 162 calculates a port position on a body surface of one amount of pneumoperitoneum (amount of pneumoperitoneum A) corresponding to a port position obtained through the port position simulation or the port position adjustment using another amount of pneumoperitoneum (amount of pneumoperitoneum B). The image generator 162 may calculate a port position on one body surface under a condition (COND2) for the stretchability of one body tissue corresponding to a port position obtained through the port position simulation or the port position adjustment using another condition (COND1) for the stretchability of another body tissue.

Next, a variation of the embodiment will be described.

The port positions may vary depending on the surgical procedure. A part or all of the planned plurality of port positions may be marked before the operation. The planned and placed port positions may be marked using a medical marker.

The port position processing unit 164 may calculate the above-described port position range before pneumoperitoneum in consideration of the error for the port positions estimated in the pneumoperitoneum simulation. The port position range before pneumoperitoneum is determined in consideration of the non-uniform extension of the subject PS or a variation in the amount of pneumoperitoneum. When the size of the derived port position range before pneumoperitoneum is more than or equal to a threshold th11, the display controller 166 may cause the display 130 to display that this port position is excluded from a preoperative marking target. When the influence (for example, a variation in port position score) of the derived port position range before pneumoperitoneum on the working area is more than or equal to a threshold th12, the display controller 166 may cause the display 130 to display that this port position is excluded from a preoperative marking target.

As a result, the user can recognize a port position before pneumoperitoneum that is most likely to become an unexpected port position by actual pneumoperitoneum during the operation. The position of this port PT is placed during the operation. When the error is large (when the port position range before pneumoperitoneum is large), a port position that is planned and placed after pneumoperitoneum may be marked.

When the size of the derived port position range before pneumoperitoneum is less than or equal to a threshold th13 which is sufficiently small, the display controller 166 may cause the display 130 to display that the error of the pneumoperitoneum simulation is sufficiently small. The display herein may be performed using, for example, character information or graphic information.

Specifically, when the size of the port position range before pneumoperitoneum is sufficiently small (that is, less than or equal to the threshold th13) in consideration of the error for the position corresponding to the injection amount of gas, the port position processing unit 164 may derive, as the port position before pneumoperitoneum, only one point (for example the central position in the port position range before pneumoperitoneum) of an average value of the port positions before pneumoperitoneum in the port position range before pneumoperitoneum derived according to the injection amount of gas. The display controller 166 may cause the display 130 to display information regarding the port position before pneumoperitoneum.

As a result, the robotically-assisted surgical device 100 can provide information regarding one port position having high accuracy while considering the error for the port position corresponding to the injection amount of gas. By checking the display indicating that the error is sufficiently small and the display of one port position before pneumoperitoneum derived from the port position range before pneumoperitoneum, the user can recognize that the port position has sufficiently small error and high accuracy. The threshold th13 may be, for example, 3 mm.

In addition, the robotically-assisted surgical device 100 may plan the port position based on 3D data of the virtual pneumoperitoneum state other than the volume data of the virtual pneumoperitoneum state. For example, the 3D data of the virtual pneumoperitoneum state may be a combination of the deformation information and the volume data of the non-pneumoperitoneum state or may be surface data of the non-pneumoperitoneum state. The surface data of the virtual pneumoperitoneum state may be generated based on the volume data of the virtual pneumoperitoneum state. The surface data of the virtual pneumoperitoneum state may be generated from the deformation information and surface data generated from the volume data of the non-pneumoperitoneum state.

The display controller 166 may visualize the error to distinguish between the error of the body surface (for example, generated by the error for the amount of gas) and the error on the body surface (for example, generated by non-uniform extension). That is, the display controller 166 may display information indicating the error of the body surface (for example, information regarding the port position range before pneumoperitoneum in consideration of the non-uniform extension) and information indicating the error on the body surface (for example, information regarding the port position range before pneumoperitoneum corresponding to the amount of gas) in different display manners. The error of the body surface indicates a difference of the body surface generated by a difference in the amount of pneumoperitoneum between virtual pneumoperitoneum and actual pneumoperitoneum. The error on the body surface indicates a difference between virtual pneumoperitoneum and actual pneumoperitoneum regarding a position to which each of corresponding points on the body surface is moved after pneumoperitoneum, the degree of the movement, and the like.

Regarding the non-uniformity of the pneumoperitoneum simulation, the deformation simulator 163 may adjust Young's modulus at each of the positions on the body surface using volume data of the past virtual pneumoperitoneum state. The volume data of the past virtual pneumoperitoneum state may be accumulated in the memory 150. That is, the deformation simulator 163 may adjust the pneumoperitoneum state of the pneumoperitoneum simulation at each of the positions on the body surface based on the past record. As a result, the robotically-assisted surgical device 100 can improve the accuracy of the pneumoperitoneum simulation. Accordingly, the robotically-assisted surgical device 100 can accurately derive the states of the subject PS before and after pneumoperitoneum and can improve the derivation accuracy of the deformation information derived based on the data before and after pneumoperitoneum. Accordingly, the robotically-assisted surgical device 100 can improve the accuracy of the port position before pneumoperitoneum.

The deformation simulator 163 may adjust Young's modulus at each of the positions on the body surface based on the property of the subject PS (for example, a patient) or the analysis result of CT data (for example, volume data). As a result, the robotically-assisted surgical device 100 can improve the accuracy of the pneumoperitoneum simulation. The property of the subject PS may be, for example, the sex, past weight, pregnancy history, skeletal frame, or subcutaneous fat. The property of the subject PS or the CT data may be stored in the memory 150.

The deformation simulator 163 may expect the amount of gas required for pneumoperitoneum (amount of pneumoperitoneum) based on the property of the subject PS or the analysis result of CT data. The property of the subject PS may be, for example, sex, vital capacity, the presence or absence of respiratory disease, skeletal frame, or resting heart rate. The property of the subject PS or the CT data may be stored in the memory 150. As a result, the robotically-assisted surgical device 100 can improve the derivation accuracy of the amount of pneumoperitoneum, and can improve the accuracy of the pneumoperitoneum simulation.

That is, based on the property of the subject PS or the analysis result of CT data, the robotically-assisted surgical device 100 may store data regarding stretchability of the skin at various positions of the subject PS in the memory 150 and may use this data for the pneumoperitoneum simulation. In addition, information regarding the property of the subject PS or the analysis result of CT data may be accumulated for each subject PS or may be accumulated for all the subjects PS without any distinction. In addition, information regarding the state of the subject PS on which pneumoperitoneum is actually performed may also be accumulated. The property of the subject PS or the analysis result of CT data and the state of the subject PS on which pneumoperitoneum is actually performed may be used as learning data. Machine learning may be performed such that the result of the pneumoperitoneum simulation based on the property of the subject PS or the analysis result of CT data approaches the state of the subject PS on which pneumoperitoneum is actually performed.

In addition, the port position before pneumoperitoneum may be determined as a range where the port PT can be provided on the body surface. That is, the port position before pneumoperitoneum may be a range of errors (the allowable error) that are allowed for the piercing of the port PT. The allowable error may be derived using various methods.

Here, the details of the allowable errors of the port position will be described.

The port position processing unit 164 derives (for examples, calculates) information (allowable error information) indicating the errors that are allowed for each of ports. The port position processing unit 164 may calculate the allowable error information based on the port position score. The port position processing unit 164 may calculate the allowable error information based on the variation of the port position score according to the movement of the port position. The allowable error may be, for example, a value that is more than a threshold th2 (for example, error: 3 mm) representing the highest level of the piercing accuracy of the port position.

The allowable error information may be displayed on the body surface of the subject PS. In this case, the projection controller 167 may project visible light representing the allowable error information to the body surface of the subject PS. In addition, the display controller 166 may display the allowable error information so as to overlap a rendering image obtained by rendering the volume data of the subject PS.

The allowable error information may be displayed as graphic information or character information. The graphic information may be displayed in a range including the allowable error that includes a port position to be pierced. This range may be a 2D range on the body surface of the subject PS. The 2D range may be a range having a circular shape (for example, an ellipse, a true circle, or other circles), a polygonal shape (for example, a rectangle, a square, a triangle, or other polygons), or other shapes. The circle or the polygon will also be referred to as “primitive shape”. The allowable error information may be displayed as other information (for example, information regarding a display manner (a display color, a display size, a display pattern, or a flashing pattern)). For example, when the allowable error of the port PT is large, the port PT may be displayed by a first color, and when the allowable error of the port PT is small, the port PT may be displayed by a second color.

The robotically-assisted surgical device 100 displays the allowable error information and the user can recognize the allowable error information and can rapidly recognize an allowable range of the piercing of a port position to be pierced. Accordingly, for example, when a spatial (for example, planar) range represented by the allowable error information is large, the user can recognize that a port can be carelessly pierced at a port position to be pierced. In addition, for example, when a spatial (for example, planar) range represented by the allowable error information is small, the user can recognize that a port is required to be accurately pierced at a port position to be pierced. Accordingly, when the robotically-assisted surgical device 100 displays, for example, allowable error information having a large allowable error, the robotically-assisted surgical device 100 can reduce a mental burden of the user who pierces the port PT. In addition, when the robotically-assisted surgical device 100 displays, for example, allowable error information having a large allowable error, the robotically-assisted surgical device 100 can reduce the number of processes required for the user who pierces the port PT to place the port, and can reduce the operative duration. In addition, when the robotically-assisted surgical device 100 displays, for example, allowable error information having a small allowable error, the robotically-assisted surgical device 100 can notify the user that high accuracy is required for the piercing of the port PT.

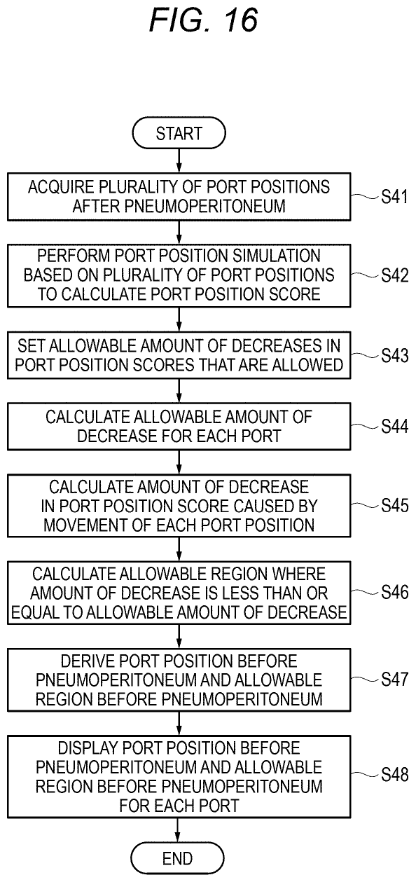

FIG. 16 is a flowchart illustrating a derivation procedure of allowable error information by the robotically-assisted surgical device 100. In FIG. 16, the acquisition of the volume data of the subject PS, the acquisition of the kinematic information of the surgical robot 300, the execution of the pneumoperitoneum simulation, and the acquisition of the information of the surgical procedure are performed in advance as illustrated in FIG. 8.

The port position processing unit 164 acquires information of a plurality of port positions (positions of piercing candidates) (port positions after pneumoperitoneum) (S41). The port position processing unit 164 performs the port position simulation to calculate the port position score based on the acquired plurality of port positions (S42). In this case, the port position processing unit 164 may calculate the port position score based on the surgical procedure, the kinematics of the surgical robot 300, the volume data of the virtual pneumoperitoneum state, and the acquired plurality of port positions. That is, here, the port position processing unit 164 may calculate the port position score for the acquired port positions.

The port position processing unit 164 acquires allowable decrease information (total allowable decrease information) regarding the degree of decreases in port position scores that are allowed for the acquired plurality of port positions (S43). The allowable decrease information may include information regarding the amount or ratio of the decreases in port position scores that are allowed for the port positions. The port position processing unit 164 may receive a user input via the UI 120 to acquire the total allowable decrease information. The port position processing unit 164 may acquire the total allowable decrease information from the memory 150. The port position processing unit 164 may acquire the total allowable decrease information from the external server via the communication unit 110.

The port position processing unit 164 acquires allowable decrease information (individual allowable decrease information) regarding the degree of a decrease in port position score that is allowed for each of the port positions (S44). The individual allowable decrease information of the respective ports PT may be the same or different. The port position processing unit 164 may derive (for example, calculate) the individual allowable decrease information based on the total allowable decrease information. In this case, the port position processing unit 164 may divide the amount of allowable decreases represented by the total allowable decrease information by the number of the ports PT to calculate the amount of an allowable decrease for each of the ports represented by the individual allowable decrease information.

In addition, the port position processing unit 164 may acquire the individual allowable decrease information without acquiring the total allowable decrease information in S33. In this case, the port position processing unit 164 may receive a user input via the UI 120 to acquire the individual allowable decrease information. The port position processing unit 164 may acquire the individual allowable decrease information from the memory 150. The port position processing unit 164 may acquire the individual allowable decrease information from the external server via the communication unit 110.

When at least one port position is moved, the port position processing unit 164 may derive (for example, calculate) decrease information indicating the degree of a decrease caused by the movement of the port position based on a port position score at the port position (a combination of port positions) before the movement and a port position score at the port position (a combination of port positions) after the movement (S45). The decrease information may include the amount or ratio of a decrease caused by the movement of the port position. In this case, the port position processing unit 164 may subtract the port position score at the port position after the movement from the port position score at the port position before the movement to calculate the amount of the decrease in port position score.

When the port position is moved, the port position processing unit 164 may calculate the amount of a change (amount of a decrease) in the port position score according to the above-described Expression 1. In this case, the amount of a decrease in the port position score before and after the movement of a port position by a predetermined distance (for example, a small distance) may correspond to the differential value F′ of the port position score F.

In addition, the movement of the port position may be performed in any direction along the body surface of the subject PS. In this case, when a body surface is a plane, the port positions before and after the movement are positioned in a 2D plane along the body surface. In addition, when the body surface includes a curved surface, the port positions before and after the movement are positioned in a 3D space.

For each of the port positions of the subject PS, the port position processing unit 164 derives a region (allowable region PR) where the decrease information satisfies the allowable decrease information (S46). In this case, the port position processing unit 164 may calculate the allowable region PR where the amount of a decrease represented by the decrease information is less than the amount of an allowable decrease represented by the allowable decrease information. The allowable region PR may be a region in a 3D space. Accordingly, a contour of the allowable region PR is a position in a 3D space that matches the amount of an allowable decrease represented by the allowable decrease information with respect to the port position.

The allowable region PR derived in S46 is the allowable region PR on the subject PS of the virtual pneumoperitoneum state. The port position processing unit 164 derives (for example, calculates) an allowable region (allowable region PR2 before pneumoperitoneum) on the subject PS of the non-pneumoperitoneum state based on the allowable region PR on the subject PS of the virtual pneumoperitoneum state and the deformation information (S47). In addition, the port position processing unit 164 derives (for example, calculates) a port position (a port position before pneumoperitoneum) on the subject PS of the non-pneumoperitoneum state based on the port position on the subject PS of the virtual pneumoperitoneum state and the deformation information (S47). The allowable region PR2 before pneumoperitoneum is an example of the allowable error information.

The port position processing unit 164 causes the display controller 166 or the projection controller 167 to display a port position before pneumoperitoneum and the allowable region PR2 before pneumoperitoneum for the port position before pneumoperitoneum for each of the ports PT (S48). In this case, the display controller 166 causes the display 130 to display the port position before pneumoperitoneum and the allowable region PR2 before pneumoperitoneum for the port position before pneumoperitoneum so as to overlap the rendering image of the subject PS for each of the ports PT. In addition, the projection controller 167 may causes the projection unit 170 to project visible light representing the port position before pneumoperitoneum and the allowable region PR2 before pneumoperitoneum for the port position before pneumoperitoneum to the body surface of the subject PS for each of the ports PT to display the port position before pneumoperitoneum and the allowable region PR2 before pneumoperitoneum. As the range of the allowable error of the port position before pneumoperitoneum, the allowable region PR2 before pneumoperitoneum may be derived and displayed.

This way, according to the embodiment, in robotic surgery, the port position into which the trocar TC is inserted is planned before pneumoperitoneum, and the planned port position is measured, placed, and marked using a skin marker or the like. After pneumoperitoneum, the port PT is pierced at the marked position, and the trocar TC is inserted into the port PT. Accordingly, port position planning or the like can be performed before pneumoperitoneum, and the operative duration can be reduced. The robotically-assisted surgical device 100 performs the port position simulation or the port position adjustment based on the volume data of the virtual pneumoperitoneum state to estimate the port position before pneumoperitoneum using the deformation information. The difficulty or the time and effort required for the operator to mark the port position as planned depends on the position of the port PT. Before the operation, the operator or the assistant can perform the planning or placement of a port position or the marking using a method that requires time and effort. In order to perform pneumoperitoneum, anesthesia is performed, and the remaining operative duration is limited. The robotically-assisted surgical device 100 can perform the planning, placement, marking, or the like of a port position before pneumoperitoneum. In addition, when the position of the port PT is measured and placed before the operation, the cleanliness of an instrument used for measurement can be set to be low. “Before pneumoperitoneum” refers to “before piercing the port PT”, and the reason for this is to prevent, for example, bacteria from entering into the body through the port PT.

Hereinbefore, various embodiments have been described with reference to the drawings. However, it is needless to say that the present disclosure is not limited to these examples. It is obvious to those skilled in the art that various changes or modifications can be conceived within the scope of the claims. Of course, it can be understood that these changes or modifications belong to the technical scope of the present disclosure

In the first embodiment, the volume data as the captured CT images are transmitted from the CT apparatus 200 to the robotically-assisted surgical device 100. Instead, the volume data may be transmitted to a network server to temporarily accumulate the data and then stored in a server or the like. In this case, as necessary, the communication unit 110 of the robotically-assisted surgical device 100 may acquire the volume data from the server or the like by wire or wirelessly, or may acquire the volume data via any storage medium (not illustrated).

In the first embodiment, the volume data as the captured CT images are transmitted from the CT apparatus 200 to the robotically-assisted surgical device 100 via the communication unit 110. This example also includes a case where the CT apparatus 200 and the robotically-assisted surgical device 100 are substantially integrated into one product. In addition, the example may also include a case where the robotically-assisted surgical device 100 is considered as a console of the CT apparatus 200.

In the first embodiment, the CT apparatus 200 captures images to generate volume data including information regarding the inside of an organism. However, another device may capture images to generate volume data. Examples of the other device include a Magnetic Resonance Imaging (MRI) device, a Positron Emission Tomography (PET) device, an angiographic device, and other modality devices. In addition, the PET device may be used in combination with other modality devices.

In the first embodiment, the surgical robot 300 is connected to the robotically-assisted surgical device 100. However, the surgical robot 300 is not necessarily connected to the robotically-assisted surgical device 100. The reason is for this is that this connection is not particularly limited as long as the kinematic information of the surgical robot 300 is acquired in advance. In addition, the surgical robot 300 may be connected after the end of the piercing of the ports. In addition, only a device that is a part of devices constituting the surgical robot 300 may be connected to the robotically-assisted surgical device 100. In addition, the robotically-assisted surgical device 100 itself may be a part of the surgical robot 300.

In the first embodiment, the surgical robot 300 is a surgical robot for minimal invasion. However, the surgical robot 300 for minimal invasion may be a surgical robot that assists laparoscopic surgery. In addition, the surgical robot 300 may be a surgical robot that assists endoscopic surgery.

In the first embodiment, the robotically-assisted surgical device 100 plans the port positions based on the volume data of the virtual pneumoperitoneum state of the subject PS, but the present disclosure is not limited thereto. For example, when the observation target is a respiratory organ or a cervical part, robotic surgery may be performed without pneumoperitoneum. That is, the robotically-assisted surgical device 100 may plan the port positions based on the volume data of the non-pneumoperitoneum state.

In the first embodiment, the subject PS is a human body but may be an animal body.

The present disclosure is also applicable to a program that implements the function of the robotically-assisted surgical device according to the first embodiment, in which the program is supplied to the robotically-assisted surgical device via a network or various storage media and is read and executed by a computer in the robotically-assisted surgical device.

As described above, the robotically-assisted surgical device 100 according to the embodiment assists minimally invasive robotic surgery by the surgical robot 300. The processing unit 160 may acquire volume data of the subject PS, and may perform a pneumoperitoneum simulation on the volume data of the subject PS to generate first deformation information including the movement of at least one point in the volume data of the non-pneumoperitoneum state, which is caused by pneumoperitoneum. The processing unit 160 may generate 3D data of a first virtual pneumoperitoneum state based on the volume data of the non-pneumoperitoneum state and the first deformation information. The processing unit 160 may derive a first planned position (for example, the position of the port PT20) that is a planned position of a port PT on a body surface of the subject PS in the 3D data of the first virtual pneumoperitoneum state. The processing unit 160 may derive a second planned position (for example, the position of the port PT10) that is a planned position of a port PT on the body surface of the subject PS in the volume data of the non-pneumoperitoneum state based on the first planned position in the first virtual pneumoperitoneum state and the first deformation information. The processing unit 160 may cause the display unit (for example, the display 130) to display information indicating the second planned position so as to overlap the volume data of the non-pneumoperitoneum state.

As a result, by using the deformation information (the first deformation information), even when the degree of elevation of abdominal wall varies depending on the pneumoperitoneum state, the abdominal cavity of the subject PS varies or the figure of elevation in the abdominal cavity varies, the robotically-assisted surgical device 100 can associate a port position on the body surface of the subject PS that is planned after pneumoperitoneum with a position corresponding to a port position on the body surface of the subject PS before pneumoperitoneum. In addition, by associating the port positions before and after pneumoperitoneum with each other, the robotically-assisted surgical device 100 can plan, before pneumoperitoneum, the position of the port PT on the body surface of the subject PS on which pneumoperitoneum is to be performed. Accordingly, the robotically-assisted surgical device 100 can plan the position of the port PT on the subject PS of the non-pneumoperitoneum state before the start of the operation and can plan the position of the port PT without considering the operative duration. Accordingly, the robotically-assisted surgical device 100 can reduce a mental burden relating to port position planning on the operator. In addition, since the robotically-assisted surgical device 100 plans the port position before the operation to remove port position planning during operation, the operative duration can be reduced, and a physical burden on the subject PS (for example, a patient) can also be reduced. In addition, by planning the port position before pneumoperitoneum, the user can measure the port position using a ruler or the like and place the port position before pneumoperitoneum. In this case, the body surface of the subject PS before pneumoperitoneum is flatter than that after pneumoperitoneum. Therefore, the user easily measures the port position by putting a ruler on the body surface and easily place the port position.

In addition, more than one first planned position may be derived or the first planned position may represent a range in the 3D data of the first virtual pneumoperitoneum state. More than one second planned position may be derived or the second planned position may represent a range in the volume data of the non-pneumoperitoneum state.

As a result, during the port position planning before pneumoperitoneum, the robotically-assisted surgical device 100 can plan a plurality of port positions to be pierced or can plan port positions in a certain range instead of points on the body surface. Accordingly, even when corresponding positions before and after pneumoperitoneum are misplaced from each other to some extent due to the error of the pneumoperitoneum simulation, the accuracy of the planned position before pneumoperitoneum is low to some extent, the placement accuracy of the port position before pneumoperitoneum is low to some extent, or the piercing accuracy for piercing the placed position after actual pneumoperitoneum is low to some extent, the robotically-assisted surgical device 100 can increase the possibility that the port position after actual pneumoperitoneum is included any point or range of the port positions planned before pneumoperitoneum.

The processing unit 160 may perform a plurality of pneumoperitoneum simulations using different pneumoperitoneum conditions to generate second deformation information including the movement of at least one point in the volume data of the non-pneumoperitoneum state, which is caused by the pneumoperitoneum. The second planned position may be derived using 3D data generated based on representative deformation information among a plurality of deformation information. In this case, the deformation information that is not used for generating the 3D data can be used for calculating the error for the planned position by comparing differences from the deformation information used for generating the 3D data.

As a result, the robotically-assisted surgical device 100 can generate 3D data of a plurality of virtual pneumoperitoneum states to plan the port position before pneumoperitoneum in consideration of the result of the plurality of pneumoperitoneum simulations, that is, in consideration of various pneumoperitoneum states of the subject PS. For example, the robotically-assisted surgical device 100 can derive the port position before pneumoperitoneum or the port position range before pneumoperitoneum corresponding to the port position after pneumoperitoneum in the volume data of the non-pneumoperitoneum state based on the planned port position after pneumoperitoneum in the 3D data of the plurality of virtual pneumoperitoneum states.

In addition, the pneumoperitoneum conditions may include an amount of pneumoperitoneum on the subject PS.

As a result, the robotically-assisted surgical device 100 can derive the port position after pneumoperitoneum in consideration of the plurality of amounts of pneumoperitoneum, that is, in consideration of the error of the pneumoperitoneum simulation corresponding to the injection amount of gas of the subject PS. The robotically-assisted surgical device 100 can derive the port position before pneumoperitoneum or the port position range before pneumoperitoneum corresponding to the port position after pneumoperitoneum and can plan the port position before pneumoperitoneum.

In addition, the pneumoperitoneum conditions may include a parameter indicating a stretchability of a body tissue of the subject PS.

As a result, the robotically-assisted surgical device 100 can derive the port position after pneumoperitoneum by considering a plurality of extension parameters, that is, by reproducing the non-uniform extension of the subject PS in the pneumoperitoneum simulation. In this case, even when the way of extension varies depending on the respective portions in the subject PS and the moving directions or moving distances of the respective points on the body surface are different, the robotically-assisted surgical device 100 can associate the respective points on the body surface before and after pneumoperitoneum with each other based on the results of the plurality of pneumoperitoneum simulations or the deformation information. The robotically-assisted surgical device 100 can derive the port position before pneumoperitoneum or the port position range before pneumoperitoneum corresponding to the port position after pneumoperitoneum and can plan the port position before pneumoperitoneum.

In addition, the processing unit 160 may acquire operation information regarding an operation of a robot arm AR of the surgical robot 300 and may acquire information of a surgical procedure for operating the subject PS. The processing unit 160 may derive the first planned position based on the operation information, the information of the surgical procedure, and the 3D data.

As a result, the robotically-assisted surgical device 100 can perform port position planning according to the robot arm AR of the surgical robot 300 or the surgical procedure before pneumoperitoneum.

In addition, the processing unit 160 may render the volume data of the non-pneumoperitoneum state to generate a rendering image. The processing unit 160 may derive a first tolerance as a range of errors that are allowed for the piercing of the port PT in the 3D data based on the 3D data, the operation information of the surgical robot 300, the surgical procedure, and the second planned position. The processing unit 160 may derive a second tolerance as a range of errors that are allowed for the piercing of the port PT in the volume data of the non-pneumoperitoneum state based on the first tolerance and the first deformation information in the first virtual pneumoperitoneum state. The processing unit 160 may cause the display unit to display information indicating the second tolerance together with the rendering image and the information indicating the second planned position.

As a result, the robotically-assisted surgical device 100 can visualize the allowable errors where misplacement from the second planned position to be pierced is allowed before pneumoperitoneum. Accordingly, the user can mark, for example, the second tolerance corresponding to the allowable errors before pneumoperitoneum.

The processing unit 160 may generate 3D data of a second virtual pneumoperitoneum state based on the volume data of the non-pneumoperitoneum state and the second deformation information. The processing unit 160 may derive a third planned position that is a planned position of a port on the body surface of the subject in the 3D data of the second virtual pneumoperitoneum state. The processing unit 160 may derive a fourth planned position that is a planned position of a port on the body surface of the subject in the volume data of the non-pneumoperitoneum state based on the third planned position and the second deformation information in the second virtual pneumoperitoneum state. The processing unit 160 may derive a planned range as a range of planned positions of ports on the body surface of the subject in the volume data of the non-pneumoperitoneum state based on the second planned position and the fourth planned position. The processing unit 160 may cause the display unit to display information indicating the planned range to so as overlap the volume data of the non-pneumoperitoneum state.

As a result, the robotically-assisted surgical device 100 can estimate, for example, port positions after pneumoperitoneum of a plurality of different virtual pneumoperitoneum state caused by pneumoperitoneum, a plurality of port positions before pneumoperitoneum corresponding to the port positions after pneumoperitoneum, and the port position range before pneumoperitoneum based on the plurality of port positions before pneumoperitoneum. As a result, even when the amount of pneumoperitoneum (an example of the pneumoperitoneum condition) changes to some extent during the operation, the robotically-assisted surgical device 100 can suppress the port positions from being misplaced at unexpected positions after pneumoperitoneum during the operation. In addition, in the robotically-assisted surgical device 100, by assuming the amount of pneumoperitoneum to have a certain range from the beginning, the influence of the variation in the amount of pneumoperitoneum on the derivation of the port positions before pneumoperitoneum can be absorbed within an error range. The same can also be applied to the case of the error generated by the non-uniform extension. That is, even when the extension state during pneumoperitoneum of the body surface of the subject PS is different from an expected state to some extent, the robotically-assisted surgical device 100 can suppress the port positions from being misplaced at unexpected positions after pneumoperitoneum during the operation. In addition, in the robotically-assisted surgical device 100, by assuming the body surface after pneumoperitoneum to have a certain range based on the assumed extension state, the influence of the variation in the extension of the body surface during pneumoperitoneum on the derivation of the port positions before pneumoperitoneum can be absorbed within an error range.

The present disclosure is useful for, for example, a robotically-assisted surgical device capable of reducing the influence of misplacement of a pre-pierced port on robotic surgery, a robotically-assisted surgery method, and a system.

What is claimed is:

1. A robotically-assisted surgical device that assists minimally invasive robotic surgery with a surgical robot,
the robotically-assisted surgical device comprising a processing unit and a display unit, wherein
the processing unit is configured to
acquire volume data of a non-pneumoperitoneum state of a subject,
perform a pneumoperitoneum simulation on the volume data of the non-pneumoperitoneum state to generate first deformation information including movement of at least one point in the volume data of the non-pneumoperitoneum state, the movement being caused by pneumoperitoneum,
generate 3D data of a first virtual pneumoperitoneum state based on the volume data of the non-pneumoperitoneum state and the first deformation information,
derive a first planned position that is a planned position of a port on a body surface of the subject in the 3D data of the first virtual pneumoperitoneum state,
derive a second planned position that is a planned position of a port on the body surface of the subject in the volume data of the non-pneumoperitoneum state based on the first planned position in the first virtual pneumoperitoneum state and the first deformation information, and
cause the display unit to visualize the volume data of the non-pneumoperitoneum state with an annotation of information indicating the second planned position.

2. The robotically-assisted surgical device according to claim 1, wherein
more than one first planned position are derived or the first planned position represents a range in the 3D data of the first virtual pneumoperitoneum state, and
more than one second planned position are derived or the second planned position represents a range in the volume data of the non-pneumoperitoneum state.

3. The robotically-assisted surgical device according to claim 1, wherein
the processing unit performs a plurality of pneumoperitoneum simulations using different pneumoperitoneum conditions to generate second deformation information including the movement of at least one point in the volume data of the non-pneumoperitoneum state, the movement being caused by the pneumoperitoneum.

4. The robotically-assisted surgical device according to claim 3, wherein
the pneumoperitoneum conditions include a parameter indicating an amount of pneumoperitoneum on the subject.

5. The robotically-assisted surgical device according to claim 3, wherein
the pneumoperitoneum conditions include a parameter indicating a stretchability of a body tissue of the subject.

6. The robotically-assisted surgical device according to claim 1, wherein
the processing unit is configured to
acquire operation information regarding operation of a robot arm of the surgical robot,
acquire information of a surgical procedure for operating the subject, and
derive the first planned position based on the operation information, the information of the surgical procedure, and the 3D data.

7. The robotically-assisted surgical device according to claim 6, wherein
the processing unit is configured to
render the volume data of the non-pneumoperitoneum state to generate a rendering image,
derive a first tolerance as a range of errors that are allowed for the piercing of the port based on the 3D data, the operation information of the surgical robot, the surgical procedure, and the first planned position,
derive a second tolerance as a range of errors that are allowed for the piercing of the port in the volume data of the non-pneumoperitoneum state based on the first tolerance and the first deformation information in the first virtual pneumoperitoneum state, and
cause the display unit to visualize the volume data of the non-pneumoperitoneum state with an annotation of information indicating the second planned position and information indicating the second tolerance.

8. The robotically-assisted surgical device according to any one of claim 3, wherein
the processing unit is configured to
generate 3D data of a second virtual pneumoperitoneum state based on the volume data of the non-pneumoperitoneum state and the second deformation information,
derive a third planned position that is a planned position of a port on the body surface of the subject in the 3D data of the second virtual pneumoperitoneum state,
derive a fourth planned position that is a planned position of a port on the body surface of the subject in the volume data of the non-pneumoperitoneum state based on the third planned position and the second deformation information in the second virtual pneumoperitoneum state,
derive a planned range as a range of planned positions of ports on the body surface of the subject in the volume data of the non-pneumoperitoneum state based on the second planned position and the fourth planned position, and
cause the display unit to visualize the volume data of the non-pneumoperitoneum state with an annotation of information indicating the planned range.

9. A robotically-assisted surgery method for assisting minimally invasive robotic surgery with a surgical robot, the robotically-assisted surgery method comprising:
acquiring volume data of a non-pneumoperitoneum state of a subject;
performing a pneumoperitoneum simulation on volume data of the non-pneumoperitoneum state to generate 3D data of a virtual pneumoperitoneum state;
generating deformation information representing a corresponding relationship between respective points in the volume data and respective points in the 3D data based on the volume data of the non-pneumoperitoneum state and the 3D data of the virtual pneumoperitoneum state;
deriving a first planned position that is a planned position of a port on a body surface of the subject in the 3D data of the virtual pneumoperitoneum state;
deriving a second planned position that is a planned position of a port on the body surface of the subject in the volume data of the non-pneumoperitoneum state based on the first planned position in the virtual pneumoperitoneum state and the deformation information; and
causing a display unit to visualize the volume data of the non-pneumoperitoneum state with an annotation of information indicating the second planned position.

10. A system, comprising:
a surgical robot; and
a robotically-assisted surgical device that assists minimally invasive robotic surgery with the surgical robot and includes a processing unit and a display unit, wherein
the processing unit is configured to
acquire volume data of a non-pneumoperitoneum state of a subject,
perform a pneumoperitoneum simulation on the volume data of the non-pneumoperitoneum state to generate first deformation information including movement of at least one point in the volume data of the non-pneumoperitoneum state, the movement being caused by pneumoperitoneum,
generate 3D data of a first virtual pneumoperitoneum state based on the volume data of the non-pneumoperitoneum state and the first deformation information,
derive a first planned position that is a planned position of a port on a body surface of the subject in the 3D data of the first virtual pneumoperitoneum state,
derive a second planned position that is a planned position of a port on the body surface of the subject in the volume data of the non-pneumoperitoneum state based on the first planned position in the first virtual pneumoperitoneum state and the first deformation information, and
cause the display unit to visualize the volume data of the non-pneumoperitoneum state with an annotation of information indicating the second planned position.

11. The system according to claim 10, wherein
more than one first planned position are derived or the first planned position represents a range in the 3D data of the first virtual pneumoperitoneum state, and
more than one second planned position are derived or the second planned position represents a range in the volume data of the non-pneumoperitoneum state.

12. The system according to claim 10, wherein
the processing unit performs a plurality of pneumoperitoneum simulations using different pneumoperitoneum conditions to generate second deformation information including the movement of at least one point in the volume data of the non-pneumoperitoneum state, the movement being caused by the pneumoperitoneum.

13. The system according to claim 12, wherein
the pneumoperitoneum conditions include a parameter indicating an amount of pneumoperitoneum on the subject.

14. The system according to claim 12, wherein
the pneumoperitoneum conditions include a parameter indicating a stretchability of a body tissue of the subject.

15. The system according to claim 10, wherein
the processing unit is configured to
acquire operation information regarding operation of a robot arm of the surgical robot,
acquire information of a surgical procedure for operating the subject, and
derive the first planned position based on the operation information, the information of the surgical procedure, and the 3D data.

16. The system according to claim 15, wherein
the processing unit is configured to
render the volume data of the non-pneumoperitoneum state to generate a rendering image,
derive a first tolerance as a range of errors that are allowed for the piercing of the port based on the 3D data, the operation information of the surgical robot, the surgical procedure, and the first planned position,
derive a second tolerance as a range of errors that are allowed for the piercing of the port in the volume data of the non-pneumoperitoneum state based on the first tolerance and the first deformation information in the first virtual pneumoperitoneum state, and
cause the display unit to visualize the volume data of the non-pneumoperitoneum state with an annotation of information indicating the second planned position and information indicating the second tolerance.

17. The system according to any one of claim 12, wherein
the processing unit is configured to
generate 3D data of a second virtual pneumoperitoneum state based on the volume data of the non-pneumoperitoneum state and the second deformation information,
derive a third planned position that is a planned position of a port on the body surface of the subject in the 3D data of the second virtual pneumoperitoneum state,
derive a fourth planned position that is a planned position of a port on the body surface of the subject in the volume data of the non-pneumoperitoneum state based on the third planned position and the second deformation information in the second virtual pneumoperitoneum state,
derive a planned range as a range of planned positions of ports on the body surface of the subject in the volume data of the non-pneumoperitoneum state based on the second planned position and the fourth planned position, and
cause the display unit to visualize the volume data of the non-pneumoperitoneum state with an annotation of information indicating the planned range.
